SEQUENCE LISTING (37 sequences)

SEQ ID NO: 1 - DNA, 21 bp, synthetic construct
  source 1..21: mol_type = other DNA
ctcccactccatgaggtattt

SEQ ID NO: 2 - DNA, 21 bp, synthetic construct
  source 1..21: mol_type = other DNA
tcccactccatgaggtatttc

SEQ ID NO: 3 - DNA, 21 bp, synthetic construct
  source 1..21: mol_type = other DNA
acgacggcaaggattacatcg

SEQ ID NO: 4 - DNA, 21 bp, synthetic construct
  source 1..21: mol_type = other DNA
agatacctggagaacgggaag

SEQ ID NO: 5 - DNA, 21 bp, synthetic construct
  source 1..21: mol_type = other DNA
cgacggcaaggattacatcgc

SEQ ID NO: 6 - DNA, 21 bp, synthetic construct
  source 1..21: mol_type = other DNA
agtgagaatctgagcttattt

SEQ ID NO: 7 - DNA, 21 bp, synthetic construct
  source 1..21: mol_type = other DNA
accgccttcgttgtcagttat

SEQ ID NO: 8 - DNA, 21 bp, synthetic construct
  source 1..21: mol_type = other DNA
agatggccattccattcttta

SEQ ID NO: 9 - DNA, 21 bp, synthetic construct
  source 1..21: mol_type = other DNA
gagtaactggcttcctttaaa

SEQ ID NO: 10 - DNA, 21 bp, synthetic construct
  source 1..21: mol_type = other DNA
catgaagtctgtcgctataaa

SEQ ID NO: 11 - DNA, 21 bp, synthetic construct
  source 1..21: mol_type = other DNA
agacattaatccctcactatt

SEQ ID NO: 12 - DNA, 21 bp, synthetic construct
  source 1..21: mol_type = other DNA
agagaatggaaagtcaaattt

SEQ ID NO: 13 - DNA, 21 bp, synthetic construct
  source 1..21: mol_type = other DNA
gcagagaatggaaagtcaaat

SEQ ID NO: 14 - DNA, 21 bp, synthetic construct
  source 1..21: mol_type = other DNA
ttcagcaaggactggtctttc

SEQ ID NO: 15 - DNA, 21 bp, synthetic construct
  source 1..21: mol_type = other DNA
tgagtatgcctgccgtgtgaa

SEQ ID NO: 16 - DNA, 21 bp, synthetic construct
  source 1..21: mol_type = other DNA
aaatacctcatggagtgggag

SEQ ID NO: 17 - DNA, 21 bp, synthetic construct
  source 1..21: mol_type = other DNA
gaaatacctcatggagtggga

SEQ ID NO: 18 - DNA, 21 bp, synthetic construct
  source 1..21: mol_type = other DNA
cgatgtaatccttgccgtcgt

SEQ ID NO: 19 - DNA, 21 bp, synthetic construct
  source 1..21: mol_type = other DNA
cttcccgttctccaggtatct

SEQ ID NO: 20 - DNA, 21 bp, synthetic construct
  source 1..21: mol_type = other DNA
gcgatgtaatccttgccgtcg

SEQ ID NO: 21 - DNA, 21 bp, synthetic construct
  source 1..21: mol_type = other DNA
aaataagctcagattctcact

SEQ ID NO: 22 - DNA, 21 bp, synthetic construct
  source 1..21: mol_type = other DNA
ataactgacaacgaaggcggt

SEQ ID NO: 23 - DNA, 21 bp, synthetic construct
  source 1..21: mol_type = other DNA
taaagaatggaatggccatct

SEQ ID NO: 24 - DNA, 21 bp, synthetic construct
  source 1..21: mol_type = other DNA
tttaaaggaagccagttactc

SEQ ID NO: 25 - DNA, 21 bp, synthetic construct
  source 1..21: mol_type = other DNA
tttatagcgacagacttcatg

SEQ ID NO: 26 - DNA, 21 bp, synthetic construct
  source 1..21: mol_type = other DNA
aatagtgagggattaatgtct

SEQ ID NO: 27 - DNA, 21 bp, synthetic construct
  source 1..21: mol_type = other DNA
aaatttgactttccattctct

SEQ ID NO: 28 - DNA, 21 bp, synthetic construct
  source 1..21: mol_type = other DNA
atttgactttccattctctgc

SEQ ID NO: 29 - DNA, 21 bp, synthetic construct
  source 1..21: mol_type = other DNA
gaaagaccagtccttgctgaa

SEQ ID NO: 30 - DNA, 21 bp, synthetic construct
  source 1..21: mol_type = other DNA
ttcacacggcaggcatactca

SEQ ID NO: 31 - protein, 9 aa, synthetic construct
VMAPRTLFL

SEQ ID NO: 32 - protein, 9 aa, synthetic construct
VMAPRTLLL

SEQ ID NO: 33 - protein, 9 aa, synthetic construct
VMAPRTLVL

SEQ ID NO: 34 - protein, 9 aa, synthetic construct
VMAPRTVLL

SEQ ID NO: 35 - protein, 9 aa, synthetic construct
VMAPRTLIL

SEQ ID NO: 36 - protein, 9 aa, synthetic construct
VMAPRALLL

SEQ ID NO: 37 - protein, 9 aa, synthetic construct
VTAPRTVLL

(19) United States
(12) Patent Application Publication
(10) Pub. No.: US 2025/0297263 A1
(43) Pub. Date: Sep. 25, 2025

(54) ONE-STEP CONSTRUCTION OF ALLOGENIC CAR-NK CELLS WITH INCREASED ANTI-TUMOR CYTOTOXICITY AND RESISTANCE TO HOST CELL REJECTION

(71) Applicants: MASSACHUSETTS INSTITUTE OF TECHNOLOGY, Cambridge, MA (US); DANA-FARBER CANCER INSTITUTE, INC., Boston, MA (US)

(72) Inventors: Jianzhu Chen, Lexington, MA (US); Fuguo Liu, Cambridge, MA (US); Rizwan Romee, Wellesley, MA (US)

(21) Appl. No.: 19/087,107

(22) Filed: Mar. 21, 2025

Related U.S. Application Data
(60) Provisional application No. 63/568,664, filed on Mar. 22, 2024.

(51) Int. Cl. C12N 15/113; C07K 14/705; C07K 14/74; C12N 5/0783; C12N 15/86

(52) U.S. Cl. CPC C12N 15/1138; C07K 14/70532; C07K 14/70539; C12N 5/0646; C12N 15/86; C12N 2310/14; C12N 2310/531; C12N 2510/00; C12N 2740/15043

(57) ABSTRACT

The present disclosure relates to RNAi DNA oligonucleotides for the suppression of an immune response and use in methods of one step construction of allogenic CAR cells capable of avoiding a host rejection.

### RELATED APPLICATIONS

This application claims priority to and the benefit of U.S. Provisional Application No. 63/568,664, filed on Mar. 22, 2024, which is incorporated by reference herein in its entirety.

### FIELD

The present disclosure relates to RNAi DNA oligonucleotides for the suppression of an immune response and use in methods of one step construction of allogenic CAR cells capable of avoiding a host rejection.

### SEQUENCE LISTING

The sequence listing associate with this application is provided in XML format in lieu of a paper copy and is hereby incorporated by reference into the specification. The name of the XML file containing the sequence listing is “MIT25757J_SeqListing.xml”. The XML file is 32,800 bytes, and created on Mar. 18, 2025, and is being submitted electronically, concurrent with the filing of this specification.

### BACKGROUND

Chimeric antigen receptor (CAR) cell therapy is one of the most promising approaches in anti-cancer treatments. The recent approvals of CD19 and BCMA targeting CAR-T cell therapies are significant breakthroughs in the field of cancer immunotherapy and have stimulated the development of genetically modified cell therapies for both hematological malignancies and solid tumors (Xie et al., 2020). To date, all six approved CAR-T cell therapies use patients' own (autologous) T cells for manufacturing (Dephil et al., 2020). However, autologous CAR cell therapies have well-known drawbacks, including 1) high cost and long vein-to-vein time due to the requirement of individualized manufacturing of CAR cells (Köhl et al., 2018; Laskowski et al., 2022); 2) the autologous CAR cells might not be effective in some patients owing to the dysfunction of source immune cells, which have been continuously stimulated by cancer cells and suppressed in the tumor microenvironment (Thommen & Schumacher, 2018); and 3) many patients have gone through multiple previous lines of treatment and are lymphopenic, leading to insufficient immune cells to yield a viable product (Laskowski et al., 2022).

The capability of using immune cells from any healthy donors (allogeneic) could potentially solve these issues. However, allogeneic cell therapies need to overcome two major issues: avoid graft-versus-host disease (GVHD) and rejection of allogeneic CAR cells. Unlike T cells, which can recognize non-self peptide/MHC and induce GVHD, natural killer (NK) cells are modulated by a set of activating and inhibitory receptors and don't mediate allo-reaction (Xie et al., 2020). Hence, NK cells have advantages over T cells in allogeneic cell therapies. However, allogeneic NK cells can be recognized and rapidly rejected by the recipient's immune system.

Human leukocyte antigen (HLA) class I molecules play central roles in modulating the T cell and NK cell responses, and it's an important consideration for allogeneic cell therapy (Montgomery et al., 2018). Classical HLA class I, including HLA-A, HLA-B, and HLA-C, (referred to herein as “HLA-ABC”) presents non-self and neoantigen-derived intracellular peptides on the cell surface to CD8+ T cells and subsequently activates their functions (Neefjes et al., 2011). However, non-classical HLA class I, HLA-E, mainly presents peptides derived from the leader sequences of HLA-ABC and HLA-G on the cell surface and interacts with CD94/NKG2 family receptors to modulate NK cell functions (Braud et al., 1998). HLA class I molecules consist of three components: the polymorphic heavy chain, the light chain β2-microglobulin (β2m), and the presented peptide (Neefjes et al., 2011). Of these three components, the β2m genes and those related to the peptide presentation-associated pathway, like transporter associated with antigen processing (TAP), are widely targeted for knocking out and knocking down HLA class I expression, and no doubt both HLA-ABC and HLA-E would be reduced. The HLA-ABC-reduced cells can escape from allogeneic T-cell killing, whereas they are susceptible to NK cell killing because NK cells recognize and kill HLA-ABC-reduced target cells (Anfossi et al., 2006). Thus, it's critical to specifically reduce surface HLA-ABC expression but maintain surface HLA-E expression.

Inhibitory immunoreceptors, also known as immune checkpoints, can suppress immune cells upon interacting with ligands on immune cells (He & Xu, 2020). The use of immune checkpoint inhibitors (ICIs) has significantly improved cancer treatment, enabling the possibility of long-term survival of patients with metastatic tumors (Johnson et al., 2022). Recently, immune checkpoint proteins were overexpressed on target cells to allow them to sustain longer in allogeneic hosts (Hu et al., 2023) showed that CD47 overexpressed primary human islets and induced pluripotent stem cells evaded NK and macrophage killing, and MHC I/II knockout and CD47 overexpressed cells survived in allogeneic recipients for a long term (Hu et al., 2023a,b). Yoshihara et al. demonstrated that overexpression of PD-L1 protected human islet-like organoids, resulting in the restoration of glucose homeostasis in immune-competent diabetic mice for 50 days (Yoshihara et al., 2018). In addition, Gornalusse et al. found that HLA-E overexpressing and HLA-ABC negative pluripotent stem cells escape from CD8+ T cell killing and lysis by NK cells in vitro and in vivo (Gornalusse et al., 2017).

Thus, there is a need for development of allogenic cell therapies that avoid rejection by host immune cells.

### SUMMARY

One embodiment described herein is an RNA interference (RNAi) DNA oligonucleotide for inhibiting expression of HLA A, B and C. In one aspect, the RNAi DNA oligonucleotide comprises siRNA or shRNA. In another aspect, the RNAi DNA oligonucleotide does not inhibit expression of HLA-E. In another aspect, the RNAi DNA oligonucleotide is shRNA.

In another aspect, the RNAi DNA oligonucleotide is 20-25 base pairs long. In another aspect, the RNAi DNA oligonucleotide comprises at least two mismatches. In one aspect, the RNAi DNA oligonucleotide comprises three mismatches. In another aspect, the mismatches are with an allele comprising HLA-A, HLA-B, HLA-C, or HLA-E. In another aspect, the mismatch is with an HLA-E allele.

In another aspect, the RNAi DNA oligonucleotide comprises a nucleotide sequence of SEQ ID NO. 1-30 and its complement. In one aspect, the RNAi DNA oligonucleotide comprises a nucleotide sequence of SEQ ID NO: 1 or SEQ ID NO: 13, and its complement.

Another aspect is a vector comprising the RNAi DNA oligonucleotide described herein. In another aspect, the vector is a lentiviral vector. In another aspect of the vector, the RNAi DNA oligonucleotide is operably linked to a promoter. In another aspect of the vector described herein, the promoter comprises an RNA polymerase III or an EF-1α promoter.

In another aspect, the vector further comprises a nucleotide sequence encoding HLA-E or variants thereof. In another aspect, the HLA-E sequence comprises a mutation in the heavy chain. In another aspect, the HLA-E mutation comprises Y84A or Y84C. In another aspect, the vector further comprises a nucleotide sequence encoding PD-L1.

Another aspect is a host cell transduced by the vector described herein. In another aspect, the host cell is a T cell, a Natural Killer (NK) cell, a macrophage, or an induced pluripotent stem cell.

Another embodiment described herein is a method of making an allogenic chimeric antigen receptor (CAR) immune cell resistant to host rejection comprising transducing an immune cell with the vector described herein. In one aspect of the method, the vector further comprises a nucleotide sequence encoding HLA-E or variants thereof. In one aspect of the method, the vector further comprises a nucleotide sequence encoding PD-L1. In one aspect of the method, the vector comprises the nucleotide sequence of SEQ ID NO. 1 or SEQ ID NO: 13, and its complement, a nucleotide sequence encoding HLA-E or variants thereof, and a nucleotide sequence encoding PD-L1, operably linked to one or more heterologous promoter. In another aspect of the method, the cell is a T cell, an NK cell, a macrophage, or an induced pluripotent stem cell.

Another embodiment described herein is a method of making an allogenic cell resistant to host rejection comprising transducing the cell with a vector comprising a nucleotide sequence encoding at least one of HLA-E or variant thereof, or PD-L1.

Another embodiment described herein is a method for increasing cytotoxicity of a CAR cell.

### BRIEF DESCRIPTION OF THE DRAWINGS

This disclosure will be more fully understood from the following detailed description, taken in conjunction with the accompanying drawings, in which:

FIG. 1 shows a schematic diagram of key molecular interactions among CD8+ T cells, NK cells and macrophages.

FIG. 2 illustrates that CD47 inhibits NK cell killing of target cells. FIG. 2A shows human NK cells express a high level of CD47 (2) vs isotype staining control (1). FIG. 2B shows the majority of human NK cells express SIRP1a (2) and isotype staining control (1). FIG. 2C shows the expression of CD47 by K562 (3) vs no staining (1) and isotype staining (2) controls. FIG. 2D is a scheme of NK cell cytotoxicity assay. FIGS. 2E and 2F show the expression of CD47 inhibits NK cell killing of K562 cells. Purified human NK cells were cocultured with parental K562 cells, CD47-expressing K562 cells without (2E) or with anti-CD47 blocking antibody and Fc blocker (2F) at the indicated E:T ratios for 4 hours. Lysis of K562 cells were quantified. Data are mean±s.d. with three technical replicates. One-way ANOVA with Tukey's multiple comparisons was performed between the indicated groups to determine the statistical difference: ****p≤0.0001, ***p≤0.001, ** p≤0.01, * p≤0.05, and ns indicates no significance.

FIG. 3 is a schematic diagram of antigen processing and presentation by MHC class I molecules.

FIG. 4 lists the most prevalent HLA-A, HLA-B, and HLA-C alleles in the global population, covering 99.99% of the human population.

FIGS. 5A-5E show the mismatches in nucleotide sequences of shRNA #1 to #5 (Table 2) to various HLA-A, HLA-B, HLA-C, and HLA-E alleles. These shRNAs have no mismatch (not shown) or one mismatch with the listed HLA-ABC alleles, but at least two mismatches with HLA-E alleles.

FIG. 6 shows the specific downregulation of HLA-ABC without affecting HLA-E expression by RNA interference (FIG. 6A). Representative flow histograms (left panel) and summary of median fluorescence intensity (MFI) (right panel) showing surface HLA-ABC expression in Jurkat T cells 48 hours post-transduction with lentivectors expressing shRNA #1 to #5 targeting HLA-ABC heavy chain (red), #6 to #8 targeting TAP1 (blue), #9 to #11 targeting TAP2 (blue), and #12 to #15 targeting B2M (green). Data in the right panel are mean±s.d., n=4. P values are shown for the indicated shRNAs as compared to the control. (FIG. 6B) shRNA #1 reduces surface HLA-A*02 expression in human NK cells. Purified NK cells from an HLA-A*02+ donor were transduced with lentivector expressing shRNA #1, and the surface HLA-A*02 was measured by flow cytometry 48 hours later. The black and 213 red histograms show untransduced and transduced NK cells stained with anti-HLA-A*02 antibody, respectively. The numbers indicate MFI. (FIG. 6C-D) shRNA #1 does not inhibit surface HLA-E expression. Jurkat T cells were either not stimulated (control) or stimulated with IFNγ (50 ng/ml) and then transduced with lentivectors expressing either shRNA #1 or #13. The surface HLA-E expression was measured by flow cytometry 48 hours post-transduction. Representative flow histograms (c) and summary of MFI of HLA-E (d). Data in e are mean±s.d., n=4. (FIG. 6E), shRNA #1 does not inhibit surface HLA-E expression in K562 cells stably expressing SCE. Shown are representative flow histograms comparing HLA-E levels in parental K562 cells (control) and K562 cells stably expressing SCE without (black) or with expression of shRNA #1 or #13.

FIG. 7 illustrates that IFNg does not upregulate (FIG. 7A) HLA-ABC or (FIG. 7B) HLA-E expression on primary human NK cells. Purified human NK cells were treated with 50 ng/ml IFNγ overnight. Expression of HLA-ABC and HLA-E was assayed by flow cytometry. Cells without staining or stained with isotype antibodies were used as control.

FIG. 8 shows a schematic representation of SCE (FIG. 8A) and three variants (FIG. 8B). FIG. 8C shows the amino acid sequences of peptides derived from the leader of the indicated HLA alleles that are tested in SCE.

FIG. 9 shows diagrams from flow cytometry staining of NK cells for HLA-A*02:01 in four donors.

FIG. 10 is a scheme of reciprocal experiments with donor 1 and donor 2 CD8+ T cells and NK cells.

FIG. 11 shows HLA-ABC reduced NK cells escape from recipient CD8+ T cell killing but subject to recipient NK cell killing with a comparison of HLA-ABC knockdown in human NK cells by shRNA #1, #2, #13, and #14. Purified NK cells from four donors were transduced with lentivectors expressing shRNA #1, #2, #13 or #14 followed by flow cytometry assay for surface HLA-ABC 48 hours later. Non-transduced NK cells were stained with isotype antibody (isotype control) or anti-HLA-ABC (control). Shown are representative flow histograms showing knockdown of surface HLA-ABC expression in NK cells from one donor (FIG. 11A) and summary of MFI of HLA-ABC on NK cells on all four donors (FIG. 11B). Data in b are mean±s.d., with NK cells from 4 different donors (n=4). Statistical comparisons are between the indicated samples and the control. (FIG. 11C) Killing of donor 2 NK cells by CD8+ T cells from donor 1 at the indicated E:T ratios. CD8+ T cells from donor 1 were first primed with mitomycin C pretreated donor 2 NK cells (with normal HLA-ABC expression) in the presence of IL-2 for 7 days. The primed CD8+ T cells were then cocultured with prelabeled NK cells from donor 2 with either normal or reduced HLA-ABC expression overnight at the indicated E:T ratios, and lysis of donor 2 NK cells was determined by flow cytometry. (FIG. 11D) Killing of donor 1 NK cells by CD8+ T cells from donor 2 in a reciprocal experiment as in 12c. (FIG. 11E) CD8+ T cells from donors 2-4 were primed with donor 1 NK cells and then cocultured with prelabeled donor 1 NK cells with either normal or reduced HLA-ABC expression. (FIG. 11F) Lysis of donor 1 NK cells are shown at the indicated E:T ratios. NK cells from donors 2-4 were cocultured with prelabeled donor 1 NK cells with either normal or reduced HLA-ABC expression. (FIG. 11G) Lysis of donor 1 NK cells is shown at the indicated E:T ratios.

FIG. 12 shows PD-L1 moderately inhibits allogeneic T cell responses. FIG. 12A shows the experimental design for assaying T cell responses to allogeneic NK cells with or without exogenous PD-L1 expression. Both CD3+ or CD8+ T cells and NK cells were purified from PBMCs of donor 1 (HLA-A*02:01+) and donor 2 (HLA-A*02:01−), and all the assays were done reciprocally. (FIG. 12B), NK cells were transduced by the lentivector expressing PD-L1 followed by flow cytometry analysis for PD-L1 expression (left panel). Transduced NK cells were purified and expanded (right panel). Black and red histograms in the right panel show PD-L1 staining of untransduced NK cells and transduced and expanded NK cells. (FIG. 12C-D), CD3+ T cells were cocultured with autologous or allogeneic NK cells without or with exogenous PD-L1 expression at 1 to 1 ratio. CD69 (FIG. 12C) and CD25 (FIG. 12D) expression by T cells were measured at 12 hours post-coculture. Shown are mean±s.d. with 4 technical replicates. (FIG. 12E) CD3+ T cells were labeled with cell tracker CTV and cocultured with autologous or allogeneic NK cells without or with exogenous PD-L1 expression at 1 to 1 ratio for 5 days. T cell proliferation as indicated by CTV dilution was assayed by flow cytometry. Shown are mean±s.d. with 4 technical replicates. (FIG. 12F) CD8+ T cells were cocultured with prelabeled autologous or allogeneic NK cells without or with exogenous PD-L1 expression overnight at E:T ratios of 0, 10 and 20. Lysis of NK cells was quantified. Data are mean±s.d. from 4 technical replicates. One-way ANOVA with Tukey's multiple comparisons was performed between different groups to determine the statistical difference. P values are shown as numbers in FIG. 12C-F.

FIG. 13 illustrates T cell response to allogeneic NK cells. FIG. 13A, shows the induction of CD69 expression by T cells in coculture with autologous or allogeneic NK cells. T cells were cocultured with either autologous or allogeneic NK cells without or with PD-L1 expression at 1:1 ratio overnight. Expression of CD69 by T cells was analyzed by flow cytometry, gating on live CD3+ T cells. FIG. 13B shows these proliferation of T cells in coculture with autologous or allogeneic NK cells. CTV labeled T cells were cocultured with either autologous or allogeneic NK cells without or with PD-L1 expression at 1:1 ratio for 5 days. Dilution of CTV was assayed by flow cytometry gating on live CD3+ T cells.

FIG. 14 shows the single-chain HLA-E inhibits NK cell killing. (FIG. 14A) Experimental schema (for A-F). (FIG. 14B) Comparison of HLA-E expression in the parental K562 cells (control) and K562 cells that were transduced to express SCE WT, SCE Y84A, and SCE Y84C. (FIG. 14C-D) SCE expression in K562 cells inhibits NK cell activation. Human NK cells were cocultured with the parental K562 cells or K562 cells expressing the three SCE variants for 4 hours. Expression of CD107a and IFNγ in NK cells was assayed by flow cytometry. Shown are percentages of CD107a+ (FIG. 14C) and IFNγ+ (FIG. 14D) NK cells following coculture with the indicated K562 cells. Data are mean±s.d. with 4 technical replicates. (FIG. 14E) SCE inhibits NK cell killing of K562 cells. Human NK cells were cocultured with the parental K562 cells or K562 cells expressing the three SCE variants at different E:T ratios for 4 hours. Lysis of target K562 cells was quantified by flow cytometry. Data are mean±s.d. with 4 technical replicates. (FIG. 14F) Comparison of SCE presenting different leader peptides in inhibiting NK cell killing. K562 cells were transduced with lentivectors expressing SCE Y84C presenting the peptides derived from the leader sequences of the indicated HLA class I alleles, and cocultured with human NK cells for 4 hours. Data are mean±s.d. with 4 technical replicates at the indicated E:T ratio. (FIG. 14G) Schema of NK cell responses to HLA-ABC reduced and SCE expressing allogeneic NK cells. NK cells were isolated from the same four donors. NK cells from donor 1 (HLA-A*02:01+) were transduced with lentivector expressing shRNA #13 alone or both shRNA #13 and SCE Y84C. Transduced NK cells (HLA-ABC-reduced or plus SCE) were sorted and expanded. NK cells from the other three donor (HLA-A*02:01−) were cocultured with donor 1 NK cells with normal (control) or reduced HLA-ABC expression (#13), or reduced HLA-ABC plus SCE expression (#13+SCE) for 6 hours. (FIG. 14H-14I) Percentages of CD107a+ and IFNγ+ donors 2-4 NK cells. Data are mean±s.d. with 3 biological replicates. (FIG. 14I) Percentages of killing of donor 1 NK cells by donors 2-4 NK cells at different E:T ratios in the presence of anti-CD47 antibody and Fc blocker. Data are mean±s.d. with 3 biological replicates. One-way ANOVA with Tukey's multiple comparisons was performed between different groups to determine the statistical difference. P values are shown as numbers in C-E, H-J.

FIG. 15 is a schematic diagram of various lentiviral constructs.

FIG. 16 shows the flow cytometry staining and gating to identify donor 1 (HLA-A*02+) untransduced and transduced NK cells and donor 2 (HLA-A*02−) T cells and NK cells.

FIG. 17 Combination of HLA-ABC reduction and expression of SCE or PD-L1 in allogeneic NK cells inhibits host T and NK cell responses. (FIG. 17A-D) Histograms show comparison of HLA-ABC expression between untransduced NK cells and shRNA #1 (FIG. 17A) or #13 (FIG. 17B) transduced NK cells, or HLA-E expression between untransduced NK cells and SCE transduced NK cells (FIG. 17C), or PD-L1 expression between untransduced NK cells and PD-L1 transduced NK 1185 cells (FIG. 17D), histogram: transduced NK cells, black histogram: untransduced NK cells. (FIG. 17E-I) NK cells from donor 1 were transduced with lentivectors expressing shRNA #1 (or #13) plus SCE Y84C and/or PD-L1. The cell mixture, containing both untransduced and transduced cells were stained with the indicated antibodies followed by flow cytometry. FIG. 17E, Flow plot showing HLA-E vs HLA-ABC expression of NK cells transduced with lentivector expressing shRNA #1 plus SCE. FIG. 17F, Flow plot showing PD-L1 vs HLA-ABC expression of NK cells transduced with lentivector expressing shRNA #1 plus PD-L1. (FIG. 17G-I), Flow plots showing HLA-ABC vs forward scatter of NK cells transduced with lentivector expressing shRNA #1 plus SCE and PD-L1. The sorted HLA-ABC negative cells are also PD-L1 and/or HLA-E positive. (FIG. 17I), (FIG. 17j) CD8+ T cell killing of allogeneic NK cells+ T cells from donors 2 and 3 were primed with untransduced NK cells from donor 1 in the presence of IL-2 for 7 days. The primed T cells were cocultured with prelabeled and untransduced (control) and transduced donor 1 NK cells overnight, and lysis of NK cells was determined by flow cytometry. Shown are representative data (mean±s.d. with 3 technical replicates) with donor 2 T cells. The numbers indicate p values of the indicated samples compared to the control at E:T ratio of 20. (FIG. 17K) NK cell killing of allogeneic NK cells. NK cells from donors 2 and 3 were cocultured with prelabeled untransduced (control) and transduced donor 1 NK cells for 6 hours, and lysis of labeled donor 1 NK cells was determined by flow cytometry. Shown are representative data (mean s.d. with 3 technical replicates) with donor 2 NK cells. The numbers indicate p values of the indicated samples compared to the control at E:T ratio of 10 or between the two indicated samples. (FIG. 17L) is the comparison of K562 cell killing by untransduced (control) and transduced NK cells. Untransduced and transduced NK cells from donor 1 were cocultured with labeled K562 cells at different E:T ratios for 4 hours, and the killing of K562 cells was determined by flow cytometry. Data are mean±} s.d. with 3 technical replicates. One-way ANOVA with Tukeys multiple comparisons was performed to compare different groups at E:T ratio of 1:1. The numbers indicate p values of the indicated sample as compared to the control at E:T=1:1.

FIG. 18 shows HLA-ABC downregulated and SCE overexpressed NK cells evade allogeneic responses in vivo (FIG. 18A) Schematic diagram of the experimental protocol. NK cells from donor 1 were transduced with lentivector expressing both shRNA #13 and SCE. The cell mixtures, containing approximately 50% transduced NK cells and 50% untransduced NK cells, were mixed with equal numbers (10×10⁶) of PBMC from donor 2, and adoptively transferred into NSG mice that were deficient in both MHC class I and II and were irradiated with 100 cGy 2 days earlier. In a reciprocal experiment, NK cells from donor 2 were transduced with lentivector expressing both shRNA #13 and SCE, mixed with equal numbers of PBMC from donor 1, and adoptively transferred into recipient mice. Cell mixture before transfer (BI), in the blood at 7, 12 and 21 days after transfer, and single cell suspension from the lung, liver, spleen, and bone marrow (BM) of recipient mice 21 days after transfer were stained for human CD45, CD56, HLA-ABC, HLA-A*02:01, HLA-E, CD107a, CD3, CD69. The relative percentages of NK cells and T cells and their expression of CD107a and CD69 from donor 1 and donor 2 were quantified. (FIG. 18B-C) Relative percentages of donor 1 NK cells (bar), transduced donor 2 NK cells (black bar), and untransduced donor 2 NK cells (open bar) among total human NK cells before transfer and day 7, 14 and 21 after transfer in the blood (FIG. 18B) or in the tissues day 21 after transfer (c). (FIG. 18D-E) (FIG. 18F-G) Relative percentages of donor 2 NK cells (bar), transduced donor 1 NK cells (black bar), and untransduced donor 1 NK cells (open bar) among total human NK cells before transfer and day 7, 14 and 21 after transfer in the blood (FIG. 18F) or in the tissues day 21 after transfer (g). (FIG. 18H-I) (FIG. 18K-J), Relative percentages of human T cells among total CD45+ human cells before transfer and day 7, 14 and 21 after transfer in the blood (j) or in the tissues day 21 after transfer (FIG. 18K). (FIG. 18L-M) Comparison of percentages of human T cells that express CD69 before transfer and day 7, 14 and 21 after transfer in the blood (FIG. 18L) or in the tissues day 21 after transfer (FIG. 18M).

FIGS. 19A-M shows a combination of HLA-ABC knockdown and exogenous PD-L1 expression in allogeneic NK cells inhibit rejection by host T cell and NK cells in mice. The experiments were done exactly the same as in FIG. 18, except lentivector expressing shRNA #1 and PD-L1 was used.

FIG. 20 shows the list of CD19 and MSLN-CAR constructs with combination of shRNA and PD-L1 or SCE expression.

FIG. 21 shows CD19-CAR and MSLN-CAR NK cells with HLA-ABC knockdown and PD-L1 or SCE expression exhibit enhanced cytotoxicity against tumor cells in vitro. FIGS. 21A-B, Expression of CD19-CAR, HLA-ABC and PD-L1 by NK cells transduced with #1+19CAR+PD-L1 (FIG. 21A) or expression of MSLN CAR, HLA-ABC and HLA-E by NK cells transduced with #1+19CAR+SCE (FIG. 21B). Red histograms, transduced NK cells; black histograms, untransduced NK cells. (FIGS. 21C-D) Comparison of lysis of Raji target cells by various CD19-CAR NK cells (FIG. 21D) or lysis of OVCAR8 target cells by various MSLN-CAR NK cells at the indicated E:T ratio. Data are mean±s.d. with 3 technical replicates from one to the two independent experiment. P values are shown between the selected samples.

FIG. 22 illustrates the PCA analysis between conditions and constructs (FIG. 22A), and between conditions and donors (FIG. 22B).

FIG. 23 Comparison of differentially expressed genes between co-cultured and resting NK cells. (FIG. 23B-C) Comparison of differentially expressed genes in resting #1+CAR+PD1240 L1 NK cells (FIG. 23B) and #1+CAR+SCE NK cells (FIG. 23C) with resting #1+CAR NK cells. (FIG. 23D-F) Comparison of differentially expressed genes in cocultured #1+CAR FIG. 21D), #1+CAR+PD-L1 (FIG. 21E), and #1+CAR+SCE (FIG. 21) NK cells with their corresponding resting cells.

FIG. 24 shows enriched pathways in #1+MSLN CAR+PD-L1 (FIG. 24A) and #1+MSLN CAR+SCE (FIG. 24B) NK cells. NK cells isolated from 4 unrelated donors were transduced with lentivectors expressing #1+MLSN CAR, #1+MLSN CAR+PD-L1, and #1+MLSN CAR+SCE. NK cells from the same donors and without transductions were used as the controls. NK cells were labeled with CTV and cocultured with OVCAR8 cells or in the media alone for 18 hours, with three replicates for each group. The cocultured NK cells were purified by sorting the CTV+ cells. RNA was isolated from purified NK cells and subjected to bulk RNA sequencing. Gene set enrichment analysis (GSEA) was performed using fgsea (v 1.29.1), and the p-values were calculated with weighted Kolmogorov-Smirnov statistic tests and adjusted using the Benjamini-Hochberg correction. Gene sets were curated from the msigDB (v 7.5.1) database. Data visualization was done with ggplot2 (v3.5.0). Cellular stress, proinflammatory, proliferation, and homeostasis related pathways are labeled in blue, yellow, red, and gray, respectively.

FIG. 25 shows (FIG. 25A) Schema of the experimental protocol. NK cells from donor 1 without transduction (group 1) or transduced with lentivectors expressing CD19 CAR (group 2), #1+CD19 CAR+PD-L1 (group 3), or #1+CD19 CAR+SCE (group 4), mixed with equal numbers of PBMC from donor 2, and adoptively transferred into recipient NSG mice that lack MHC class I and II and had been injected (IV) with 0.5 million Raji-luciferase two days earlier. (FIG. 25B-C) Tumor burden was assessed on day 7, day 14, and day 21 by BLI (FIG. 25B) and presented as the normalized intensity of BLI (FIG. 25C). Groups 1, 2, 3, and 4 mice are labeled in black, blue, orange, and red, respectively. Data are mean±s.d. (FIG. 25D) Kaplan-Meier curves showing survival of mice. The numbers indicate the p values between the indicated two groups.

FIG. 26 shows Relative percentages of NK cells and T cells and their expression of CD107a and CD69 in blood and tissues. Cell mixture before transfer (BI), PBMCs 14 days after transfer and at the endpoint (EP), and single-cell suspension from the lung, liver, spleen, and bone marrow (BM) of recipient mice at the endpoint were stained for human CD45, CD56, HLA-ABC, HLA-A*02:01, HLA-E, CD107a, CD3, CD69. FIGS. 26A-D, Relative percentages of donor 2 NK cells (bar), donor 1 transduced NK cells (black bar), and donor 1 untransduced NK cells (open bar) before infusion (BI), in the blood at day 14 after transfer and at the endpoint, and in the tissues at the endpoint in group 1 (FIG. 26A), group 2 (FIG. 26B), group 3 (FIG. 26C), and group 4 (FIG. 26D). FIG. 26E-H, Relative percentages of donor 1 T cells before infusion (BI), in the blood at day 14 after transfer and at the endpoint, and in the tissues at the endpoint in group 1 (FIG. 26E), group 2 (FIG. 26E), group 3 (FIG. 26E), and group 4 (FIG. 26E).

FIG. 27 shows Percentages of CD107a+ donor 2 NK cells (bar), donor 1 transduced NK cells (black bar), and donor 1 untransduced NK cells (open bar) in blood (FIG. 27A) at day 14 after transfer and in spleen (FIG. 27B) at the endpoint. FIG. 27C-D, Percentages of CD69+ donor 2 T cells in the blood (FIG. 27C) at day 14 and in spleen (FIG. 27D) at the endpoint. Four mice each for group 1 and group 2 and five mice each for group 3 and group 4 at day 14 after transfer. Three mice for each group at the endpoint. The p values are shown for the indicated comparisons.

FIG. 28 shows FIG. 28A-F, Comparison of percentages of donor 2 NK cells (bar), donor 1 transduced CAR NK cells (black bar), and donor 1 untransduced NK cells (open bar) that express CD107a in blood on day 14 (FIG. 28A), at the endpoint (FIG. 28B), and in lung (FIG. 28C), liver (FIG. 28D), spleen (FIG. 28E), and bone marrow (FIG. 28F) among four groups. FIG. 28G-L, Comparison of percentages of donor 2 T cells that express CD69 in blood on day 14 (FIG. 28G), at the endpoint (FIG. 28H), and in lung (FIG. 28I), liver (FIG. 28J), spleen (FIG. 28K), and bone marrow (FIG. 28L) among four groups.

### DETAILED DESCRIPTION

Chimeric antigen receptor (CAR)-armed T cells (CAR-T) and natural killer cells (CAR-NK) have demonstrated great efficacy in treating liquid tumors and are being developed for treating solid tumors. Typically, a patient's own T cells or NK cells are harvested and transduced with lentivectors encoding CAR, expanded in vitro, and then transferred back into the same patients for cancer therapy. There is a great need to transduce allogeneic T cells and NK cells from healthy donors for cancer therapy because T cells and NK cells from healthy donors are easier to source and more active and can be prepared as “off-the-shelf” products. However, allogeneic T cells and NK cells are rejected by host immune cells, including T cells and NK cells. To suppress the rejection of allogenic CAR-T and CAR-NK cells, one common approach is to inhibit MHC class I expression on allogenic T and NK cells

Described herein are novel RNAi DNA oligonucleotides for suppression of HLA-ABC, a method of making an allogenic chimeric antigen receptor (CAR) immune cell resistant to host cell rejection, and a method for increasing cytotoxicity of a chimeric antigen receptor (CAR) cell.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

It is to be understood that the methods described in this disclosure are not limited to particular methods and experimental conditions disclosed herein; as such methods and conditions may vary. It is also to be understood that the terminology used herein is for the purpose of describing particular embodiments only, and is not intended to be limiting. Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., Ny, Ny. (1987-2008), including all supplements, Molecular Cloning: A Laboratory Manual (Fourth Edition) by MR Green and J. Sambrook.

Generally, nomenclature used in connection with cell and tissue culture, molecular biology, immunology, microbiology, genetics and protein and nucleic acid chemistry and hybridization described herein are those well-known and commonly used in the art. The methods and techniques provided herein are generally performed according to convention methods well known in the art and as described in various general and more specific references that are cited and discussed throughout the present specification unless otherwise indicated.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting.

Unless otherwise defined, all terms of art, notations, and other scientific terms or terminology used herein are intended to have the meanings commonly understood by those of skill in the art to which the application pertains. In some cases, terms with commonly understood meanings are defined herein for clarity and/or for ready reference, and the inclusion of such definitions herein should not necessarily be construed to represent a substantial difference over what is generally understood in the art. The following references provide one of skill with a general definition of many of the terms used in the instant disclosure: Singleton et al., Dictionary of Microbiology and Molecular Biology (2nd ed. 1994): The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991).

As used herein, the following terms have the meanings as ascribed to them below, unless specified otherwise.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “antisense oligonucleotide” means a plurality of linked nucleosides, at least a portion of which, is complementary to a target nucleic acid to which it is capable of hybridizing, resulting in at least one antisense activity. In one aspect described herein, oligonucleotides comprise one or more of deoxyribonucleosides (DNA) and/or ribonucleosides (RNA). As used herein a “nucleotide” means a nucleoside further comprising a phosphate linking group. The nucleotides described herein may be found in both DNA and RNA, and may be referred to by their full name, or single letter abbreviation, all interchangeably.

The term “about” or “approximately” means within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. In another example, the amount “about 10” includes 10 and any amounts from 9 to 11. In yet another example, the term “about” in relation to a reference numerical value may also include a range of values plus or minus 10%, 9%, 8%, 7%, 6%, 5%, 4%, 3%, 2%, or 1% from that value. Alternatively, particularly with respect to biological systems or processes, the term “about” may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “chimeric antigen receptor cell” means any immune cell which is genetically modified to express a chimeric receptor with specificity for a particular antigen. Exemplary immune cells include, but are not limited to, T-cells, Natural Killer (NK) cells, pluripotent stem cells or macrophages. Cells may have specificity for any known antigen, including but not limited to, CD19, BCMA, and Mesothelin specific CAR cells. CARs may include (1) an extracellular antigen-binding motif (e.g., single-chain variable fragment (scFv) antibody), (2) linking/transmembrane motifs, and (3) an intracellular domain, including a costimulatory domain and an activity domain (e.g., CD137 (4-IBB) and CD247 (CD3ζ)-derived costimulatory domain and an activity domain, respectively). In aspects, the CAR expresses an anti-mesothelin antibody or fragment thereof or an anti-CD19 antibody or fragment thereof (e.g., Intl. Pat. App. No. PCT/US2023/066794, filed May 9, 2023, incorporated herein by reference in its entirety; US. Pat. App. No. U.S. Ser. No. 17/910,776, filed Jan. 8, 2021, incorporated herein by reference in its entirety).

As used herein, the term “complementary” in reference to oligonucleotides means the capacity of the oligonucleotide to hybridize to another oligonucleotide compound or region via established Watson-Crick nucleotide base pairing rules, resulting in hybridization. Some mismatches are tolerated, thus in one aspect, antisense oligonucleotides may be 70% complementary. In other aspects, antisense oligonucleotides may be 80% complementary. In some aspects, antisense oligonucleotides may be 90% complementary. In some aspects, antisense oligonucleotides may be 95% complementary. In yet other aspects, antisense oligonucleotides may be 99% complementary. In yet other aspects, antisense oligonucleotides may be 100% complementary.

The term “comprise”, “comprises”, and “comprising” will be understood to imply the inclusion of a stated step or element or group of steps or elements but not the exclusion of any other step or element or group of steps or elements. By “consisting of” is meant to include, and be limited to, whatever follows the phrase “consisting of.” Thus, the phrase “consisting of” indicates that the listed elements are required or mandatory, and that no other elements may be present. By “consisting essentially of” is meant including any elements listed after the phrase, and be limited to other elements that do not interfere with or contribute to the activity or action specified in the disclosure for the listed elements.

An “expression vector” or “vector” is any genetic element, e.g., a plasmid, a mini-circle, a nanoplasmid, chromosome, virus, transposon, behaving either as an autonomous unit of polynucleotide replication within a cell. (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, transposons, bacteriophages, cosmids or virus based vectors. Vectors may contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors may be capable of directly expressing nucleic acid sequence products encoded therein without ligation or integration of the vector into host cell DNA sequences. In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11:1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP.

The term “promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. The term “promoter activity” and its grammatical equivalents as used herein refer to the extent of expression of nucleotide sequence that is operably linked to the promoter whose activity is being measured. Promoter activity may be measured directly by determining the amount of RNA transcript produced, for example by Northern blot analysis or indirectly by determining the amount of product coded for by the linked nucleic acid sequence, such as a reporter nucleic acid sequence linked to the promoter.

The term “operably linked” as used herein refers to refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner, which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers may be located upstream, downstream or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

“Polynucleotide” or “oligonucleotide” as used herein refers to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs.

As used herein “RNAi DNA oligonucleotides” are double stranded DNA molecules for expression of an RNAi transcription product capable of sequence-specific suppression of gene expression at either the transcriptional or translational level. Exemplary RNAi DNA oligonucleotides described herein include, but are not limited to, small interfering RNA (siRNA) or short hairpin RNA (shRNA), both for expression of siRNA or shRNA transcription products. shRNA sequences may form stem-loops structure of 15 to 30 base pairs (bp) region of double-stranded RNA bridged by a single-stranded RNA “loop”. shRNAs may be subsequently cleaved at the loop by the nuclease Dicer in the cytoplasm, and enter the RISC to direct cleavage and subsequent degradation of complementary mRNA. In one aspect, an RNAi DNA oligonucleotide described herein comprises an siRNA or an shRNA. In another aspect, the RNAi DNA oligonucleotide may be about 15 to 22 bp, about 18 to 28 bp, about 19 to 27 bp, about 20 to 30 bps. In another aspect described herein is an RNAi DNA oligonucleotide means for inhibiting expression of HLA-A, HLA-B and HLA-C.

Unless otherwise stated, nucleic acid sequences in the text of this specification are given, when read from left to right, in the 5′ to 3′ direction.

As used herein, the phrase “variant” when used with reference to a nucleic acid or polypeptide refers to a nucleic acid or polypeptide that differs from the referenced nucleic acid or polypeptide (for example, differing by at least one amino acid substitution from a wild-type sequence) but possesses the primary function of the referenced polypeptide. For example, a functional variant of a polypeptide that serves as a transmembrane domain is a fragment of that polypeptide that also serves as a transmembrane domain. When used with reference to a nucleic acid, the phrase “variant” refers to a nucleic acid that differs from the referenced nucleic acid but encodes a polypeptide having the same primary function as the polypeptide encoded by the referenced nucleic acid.

The terms “transfection,” “transformation,” “nucleofection,” or “transduction” as used herein refer to the introduction of one or more exogenous polynucleotides into a host cell or organism by using physical, chemical, and/or electrical methods. The nucleic acid sequences and vectors disclosed herein may be introduced into a cell or organism by any such methods, including, for example, by electroporation, calcium phosphate co-precipitation, strontium phosphate DNA co-precipitation, liposome mediated-transfection, DEAE dextran mediated-transfection, polycationic mediated-transfection, tungsten particle-facilitated microparticle bombardment, viral, and/or non-viral mediated transfection. In some cases, the method of introducing nucleic acids into the cell or organism involve the use of viral, retroviral, lentiviral, or transposon, or transposable element-mediated (e.g., Sleeping Beauty) vectors. In another aspect described herein is a vector means for inhibiting expression of HLA-A, HLA-B and HLA-C.

“Polypeptide”, “peptide”, and their grammatical equivalents as used herein refer to a polymer of amino acid residues. The polypeptide may optionally include glycosylation or other modifications typical for a given protein in a given cellular environment. Polypeptides and proteins disclosed herein (including functional portions and functional variants thereof) may comprise synthetic amino acids in place of one or more naturally-occurring amino acids. Such synthetic amino acids are known in the art, and include, for example, aminocyclohexane carboxylic acid, norleucine, α-amino n-demayoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine p-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,β-diaminopropionic acid, homophenylalanine, and α-tert-butylglycine. The present disclosure further contemplates that expression of polypeptides or proteins described herein in an engineered cell may be associated with post-translational modifications of one or more amino acids of the polypeptide or protein. Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

The terms “identical” and its grammatical equivalents as used herein or “sequence identity” in the context of two nucleic acid sequences or amino acid sequences of polypeptides refer to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244(1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment may also be performed by inspection and manual alignment. In one class of embodiments, the polypeptides herein are at least 80%, 85%, 90%, 98% 99% or 100% identical to a reference polypeptide, or a fragment thereof, e.g., as measured by BLASTP (or CLUSTAL, or any other available alignment software) using default parameters. Similarly, nucleic acids may also be described with reference to a starting nucleic acid, e.g., they may be 50%, 60%, 70%, 75%, 80%, 85%, 90%, 98%, 99% or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, the percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 95% sequence identity with a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In some embodiments, the substantial identity exists over a region of the sequences that is at least about 50 residues in length, over a region of at least about 100 residues, and in some embodiments, the sequences are substantially identical over at least about 150 residues. In some embodiments, the sequences are substantially identical over the entire length of the coding regions.

“Homology” is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95%, or 99% or more may also be used to establish homology. Methods for determining sequence identity percentages (e.g., BLASTP and BLASTN using default parameters) are described herein and are generally available. Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules may be termed “homologs.” For example, any naturally occurring proteins may be modified by any available mutagenesis method. When expressed, this mutagenized nucleic acid encodes a polypeptide that is homologous to the protein encoded by the original nucleic acid.

Also contemplated and included herein are nucleic acid molecules that hybridize to the disclosed sequences. Hybridization conditions may be mild, moderate, or stringent, as is warranted. Appropriate stringency conditions which promote DNA hybridization, for example, 6× sodium chloride/sodium citrate (SSC) at about 450 C, followed by a wash of 2×SSC at 50° C., are known or may be found in Current Protocols in Molecular Biology, John Wiley & Sons, N.Y. (1989), 6.3.1-6.3.6. “Stringent hybridization conditions” are those in which the salt concentration is less than about 1.5 M Na+ ion, typically about 0.01 to 1.0 M Na+ ion concentration (or other salts) at pH 7.0 to 8.3, and the temperature is at least about 30° C. for short sequences (such as, for example, 10 to 50 nucleotides) and at least about 60° C. for longer sequences (such as, for example, greater than 50 nucleotides). Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In one aspect described herein, a “host cell” includes cells transfected, infected, or transduced in vivo, ex vivo, or in vitro with a recombinant vector or a polynucleotide of the disclosure. Host cells may include packaging cells, producer cells, and cells infected with viral vectors. In some embodiments, host cells infected with viral vector of the disclosure are administered to a subject in need of therapy. In certain embodiments, the term “target cell” is used interchangeably with host cell and refers to transfected, infected, or transduced cells of a desired cell type. In some embodiments, the target cell is a T cell, an NK cell or an induced pluripotent stem cell (iPSC) cell. Host cells may be either autologous or allogenic. In some aspects, allogenic host cells may be obtained from a pool of healthy donors cells, including but not limited to, peripheral blood mononuclear cells (PMBCs), umbilical cord blood stem cells (UCBs), or induced pluripotent stem cell (iPSC) cells. In one aspect described herein, the host cell may be transduced with any of the vectors described herein. In another aspect is a host cell means for increased CAR cytotoxicity.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Suppression of the immune response to allogenic CAR-T (and CAR-NK) therapy may be facilitated by suppression of HLA-ABC complex proteins. However, the immune response is multi-tiered, and includes not only T-cells, but NK cells and macrophages. Thus, while suppression of HLA-ABC may avoid the immune response from T-cells, attack from other cell types may be contemplated. NK cells in particular recognize and kill HLA-ABC-reduced cells. Thus, maintaining expression of HLA-E specifically may be needed to avoid an immune response from NK cells. Furthermore, immune checkpoint PD-L1 has been found to also suppress an immune response. Thus, maintaining or enhancing expression of HLA-E and PD-L1, in addition to inhibiting expression of HLA-ABC may provide a multi-tiered approach to avoiding the immune response for any allogenic CAR molecule.

Thus, described herein are novel RNAi DNA oligonucleotides for suppression of HLA-ABC, a method of making an allogenic chimeric antigen receptor (CAR) immune cell resistant to host cell rejection, and a method for increasing cytotoxicity of a chimeric antigen receptor (CAR) cell.

One embodiment described herein is an RNA interference (RNAi) oligonucleotide for inhibiting expression of HLA A, B and C (HLA-ABC). In one aspect of the embodiment, the RNAi DNA oligonucleotide comprises an siRNA or an shRNA. In another aspect, the RNAi DNA oligonucleotide comprises an short hairpin RNA (shRNA) that is conserved in common HLA-ABC alleles but has two nucleotide mismatches in the corresponding region in major HLA-E alleles. The shRNA is designed to specifically inhibit gene expression of HLA-ABC on immune cells, and thus reduce or eliminate surface HLA-ABC expression. The shRNA does not inhibit expression of HLA-E expression. Thus the, HLA-ABC reduced immune cells may escape allogeneic CD8+ T cell killing, HLA-ABC-specifically reduced, along with PD-L1 or HLA-E overexpressed immune cells may survive and escape allogeneic responses and controlled tumor progression in vivo. In another aspect the shRNA can also be applied to specifically knock down HLA-ABC in T cells, NK cells, induced pluripotent stem cells, or macrophages, or other cell types.

In one aspect, the RNAi DNA oligonucleotide is about, 15-25 bp long, about 18-28 bp long or about 20-25 bp long. In another aspect, the RNAi DNA oligonucleotide comprises at least one mismatch. In another aspect, the RNAi DNA oligonucleotide comprises at least two mismatches. In another aspect, the RNAi DNA oligonucleotide comprises at least three mismatches. In another aspect, the mismatches are with an allele comprising HLA-A, HLA-B, HLA-C, or HLA-E. In another aspect, the RNAi DNA oligonucleotide described herein comprises a nucleotide sequence of SEQ ID NO. 1-30 and a complement thereof. In another aspect, the RNAi DNA oligonucleotide comprises a nucleotide sequence of SEQ ID NO. 1 and its complement. In one aspect, the RNAi DNA oligonucleotide comprises the nucleotide sequence of SEQ ID NO: 13 and its complement. Table 1 describes exemplary RNAi DNA oligonucleotide molecules as described herein.

TABLE 1
Exemplary RNAi DNA oligonucleotide sequences

| | |
|---|---|
| SEQ ID NO: 1 | CTCCCACTCCATGAGGTATTT |
| SEQ ID NO: 2 | TCCCACTCCATGAGGTATTTC |
| SEQ ID NO: 3 | ACGACGGCAAGGATTACATCG |
| SEQ ID NO: 4 | AGATACCTGGAGAACGGGAAG |
| SEQ ID NO: 5 | CGACGGCAAGGATTACATCGC |
| SEQ ID NO: 6 | AGTGAGAATCTGAGCTTATTT |
| SEQ ID NO: 7 | ACCGCCTTCGTTGTCAGTTAT |
| SEQ ID NO: 8 | AGATGGCCATTCCATTCTTTA |
| SEQ ID NO: 9 | GAGTAACTGGCTTCCTTTAAA |
| SEQ ID NO: 10 | CATGAAGTCTGTCGCTATAAA |
| SEQ ID NO: 11 | AGACATTAATCCCTCACTATT |
| SEQ ID NO: 12 | AGAGAATGGAAAGTCAAATTT |
| SEQ ID NO: 13 | GCAGAGAATGGAAAGTCAAAT |
| SEQ ID NO: 14 | TTCAGCAAGGACTGGTCTTTC |
| SEQ ID NO: 15 | TGAGTATGCCTGCCGTGTGAA |
| SEQ ID NO: 16 | AAATACCTCATGGAGTGGGAG |
| SEQ ID NO: 17 | GAAATACCTCATGGAGTGGGA |
| SEQ ID NO: 18 | CGATGTAATCCTTGCCGTCGT |
| SEQ ID NO: 19 | CTTCCCGTTCTCCAGGTATCT |
| SEQ ID NO: 20 | GCGATGTAATCCTTGCCGTCG |
| SEQ ID NO: 21 | AAATAAGCTCAGATTCTCACT |
| SEQ ID NO: 22 | ATAACTGACAACGAAGGCGGT |
| SEQ ID NO: 23 | TAAAGAATGGAATGGCCATCT |
| SEQ ID NO: 24 | TTTAAAGGAAGCCAGTTACTC |
| SEQ ID NO: 25 | TTTATAGCGACAGACTTCATG |
| SEQ ID NO: 26 | AATAGTGAGGGATTAATGTCT |
| SEQ ID NO: 27 | AAATTTGACTTTCCATTCTCT |
| SEQ ID NO: 28 | ATTTGACTTTCCATTCTCTGC |
| SEQ ID NO: 29 | GAAAGACCAGTCCTTGCTGAA |
| SEQ ID NO: 30 | TTCACACGGCAGGCATACTCA |

In another aspect described herein, the RNAi DNA oligonucleotide may be engineered into a vector. Exemplary vectors include but are not limited to, lentivirus vectors, retroviral vector, plasmids, AAV vectors, but any suitable vector may be used as described herein. In one aspect, the RNAi DNA oligonucleotide is integrated into a suitable vector under the control of an appropriate promoter. Exemplary promoters include but are not limited to, RNA polymerase III and EF-1α promoters. Single promoters or multiple promoters may be used in series, or used separately each for control of separate elements. Thus in another aspect described herein the RNAi DNA oligonucleotide is operably linked to a promoter. In addition, in another aspect, the same vector may further comprise a sequence for enhanced expression of HLA-E and/or PD-L1 in addition to the sequence for the RNAi DNA oligonucleotide. Wild-type HLA-E may be used, or a modified HLA-E, in which mutations are introduced to further improve function or expression. In one aspect, the HLA-E sequence comprises an HLA-E variant, i.e. an HLA-E with a mutation. In another aspect, the mutation is a Y84A or Y84C mutation (see for exemplary mutations, Hanson et al., 2010, incorporated by reference herein only with regard to such background teaching). Without being bound by any theory, it is believed that HLA-E variants contribute to enhanced cytotoxicity of the CAR cell. Similarly, wild-type PD-L1 or a modified PD-L1 may also be introduced for further avoidance of the immune response.

Thus, the transduction into allogenic immune cells of a single vector for expression of a CAR that also includes an RNAi DNA oligonucleotide sequence for inhibition of expression of HLA-ABC, and codes for simultaneous expression for HLA-E and/or PD-L1, results in a “one-step” method for producing allogenic CAR-cells for use in therapy. This single-step method allows for the use of allogenic cells with reduced immunogenicity and risk of rejection to a patient, creating the potential of “off-the shelf” CAR-cell therapies. In an aspect, a single vector comprises both (1) a CAR construct for expression and (2) an RNAi DNA oligonucleotide for RNAi expression. Various CAR constructs are contemplated, such as a CAR construct for expressing anti-CD19 or a CAR construct for expressing anti-mesothelin (e.g., Intl. Pat. App. No. PCT/US2023/066794, filed May 9, 2023, incorporated herein by reference in its entirety; US. Pat. App. No. U.S. Ser. No. 17/910,776, filed Jan. 8, 2021, incorporated herein by reference in its entirety).

Thus, another embodiment described herein is a method of making an allogenic CAR cells resistant to host rejection comprising transducing the immune cell with the vectors described herein. Such cells will thus express not only the CAR, but also knock out expression of HLA-ABC, and maintain or enhance expression of HLA-E and/or PD-L1 to avoid the host immune response.

Another embodiment described herein is method of making an allogenic cell resistant to host rejection comprising transducing a cell with a vector comprising a nucleotide sequence encoding for HLA-E or a variant thereof, a nucleotide sequence encoding for PD-L1, or both.

Another embodiment described herein is a method for increasing cytotoxicity of a CAR-cell comprising transducing the immune cell with the vectors described herein.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

### Examples

To comprehensively evaluate the suppression of pMHC expression in combination with exogenous expression of PD-L1 and/or HLA-E in overcoming allo-rejection of CAR-NK cells by both host T and NK cells, shRNA was chosen to develop knockdown human HLA-A, HLA-B, and HLA-C (collectively referred to as HLA-ABC) with the goal to integrate the shRNA into the same CAR lentivector for one-step construction of allogeneic CAR-NK cells, without needing multiple steps of genetic modifications assuming that shRNA-mediated knockdown is less complete than CRISPR-mediated knockout, simultaneous expression of HLA-E and/or PD-L1 would further enhance allogeneic NK cell resistance to rejection by the host CD8+ T cells and NK cells. Specifically, a novel shRNA was identified that knocks down HLA-ABC expression without decreasing the HLA-E expression and leads to a significant abrogation of host CD8+ T cell- and NK cell-mediated rejection. Combination of shRNA-mediated HLA-ABC knockdown and exogenous expression of PD-L1 and/or HLA-E in NK cells dramatically reduced allo-rejection by the host immune system in vitro and in vivo. Furthermore, shRNA, PD-L1 or HLA-E and a CAR were engineered into the same lentivector for one-step construction of allogeneic CAR-NK cells, which were effective in CAR-dependent tumor control and resistant to allo-rejection by host T and NK cells. Unexpectedly, exogenous PD-L1 and HLA-E expression significantly enhanced the anti-tumor activity of NK and CAR-NK cells, likely due to reduced exhaustion and upregulation of genes involved in cytotoxicity and proliferation.

### Materials and Methods

### Animals and Cell Lines

The NSG MHC I/II DKO mice combined the features of severe combined immune deficiency (scid), IL-2 receptor gamma chain deficiency, and MHC I and MHC II deficiency were purchased from The Jackson Laboratory and were maintained under specific pathogen-free conditions at Massachusetts Institute of Technology. All the mouse studies were approved by the Massachusetts Institute of Technology (Protocol ID: 0322-021-25) and performed in accordance with the guidelines from the Committee on Animal Care of MIT. The HEK293T and OVCAR8 cells were cultured in Dulbecco's modified Eagle's medium (DMEM) supplemented with fetal bovine serum (FBS) (10% v/v, Gibco), penicillin/streptomycin (1% v/v, Gibco), and 2 mM L-glutamine. Suspension-adapted HEK293T cells were cultured in FreeStyle™ 293 medium (ThemoFisher Scientific) supplemented with penicillin/streptomycin (1% v/v, Gibco). Jurkat T cells and Raji cells were cultured in RPMI-1640 medium supplemented with 10% FBS, 1% penicillin/streptomycin, and 2 mM L-glutamine.

Design of sRNA

For shRNAs targeting B2M, transporter associated with antigen processing (TAP) 1, and TAP2 mRNA sequences, the mRNAs were first obtained from the National Center for Biotechnology Information (NCBI). After that, shRNAs were predicted using the Genetic Perturbation Platform developed by Broad Institute (https://portals.broadinstitute.org/gpp/public/seq/search) with the default parameters. At least three top-ranked shRNAs were randomly selected for each target gene for further investigation. Table 2 describes exemplary shRNAs as used herein.

TABLE 2
Exemplary shRNA oligonucleotide sequences

| | | | SEQ | | SEQ |
|---|---|---|---|---|---|
| | | | ID | | ID |
| Target | shRNA | Sense Strand | NO: | Antisense Strand | NO: |
| HLA- | #1 | CTCCCACTCCATGA | 1 | AAATACCTCATGGAGT | 16 |
| ABC | | GGTATTT | | GGGAG | |
| | #2 | TCCCACTCCATGAG | 2 | GAAATACCTCATGGA | 17 |
| | | GTATTTC | | GTGGGA | |
| | #3 | ACGACGGCAAGGA | 3 | CGATGTAATCCTTGCC | 18 |
| | | TTACATCG | | GTCGT | |
| | #4 | AGATACCTGGAGA | 4 | CTTCCCGTTCTCCAGG | 19 |
| | | ACGGGAAG | | TATCT | |
| | #5 | CGACGGCAAGGAT | 5 | GCGATGTAATCCTTGC | 20 |
| | | TACATCGC | | CGTCG | |
| TAP1 | #6 | AGTGAGAATCTGA | 6 | AAATAAGCTCAGATTC | 21 |
| | | GCTTATTT | | TCACT | |
| | #7 | ACCGCCTTCGTTGT | 7 | ATAACTGACAACGAA | 22 |
| | | CAGTTAT | | GGCGGT | |
| | #8 | AGATGGCCATTCCA | 8 | TAAAGAATGGAATGG | 23 |
| | | TTCTTTA | | CCATCT | |
| TAP2 | #9 | GAGTAACTGGCTTC | 9 | TTTAAAGGAAGCCAG | 24 |
| | | CTTTAAA | | TTACTC | |
| | #10 | CATGAAGTCTGTCG | 10 | TTTATAGCGACAGACT | 25 |
| | | CTATAAA | | TCATG | |
| | #11 | AGACATTAATCCCT | 11 | AATAGTGAGGGATTA | 26 |
| | | CACTATT | | ATGTCT | |
| B2M | #12 | AGAGAATGGAAAG | 12 | AAATTTGACTTTCCAT | 27 |
| | | TCAAATTT | | TCTCT | |
| | #13 | GCAGAGAATGGAA | 13 | ATTTGACTTTCCATTC | 28 |
| | | AGTCAAAT | | TCTGC | |
| | #14 | TTCAGCAAGGACT | 14 | GAAAGACCAGTCCTT | 29 |
| | | GGTCTTTC | | GCTGAA | |
| | #15 | TGAGTATGCCTGCC | 15 | TTCACACGGCAGGCAT | 30 |
| | | GTGTGAA | | ACTCA | |

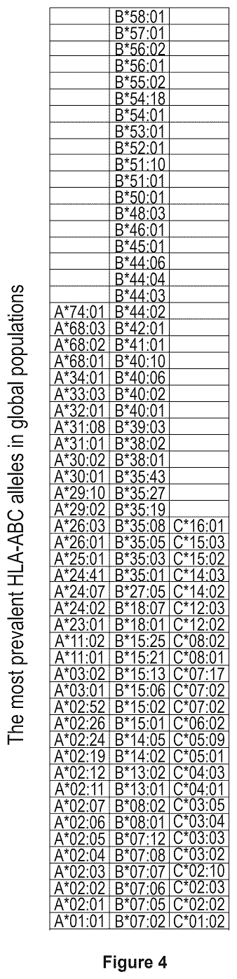

For designing shRNAs targeting HLA-ABC heavy chain specific regions that are common to prevalent HLA-ABC alleles but have at least two nucleotide mismatches compared to HLA-E*01:01 and HLA-E*01:03, the prevalent HLA-ABC alleles were first obtained in global populations from the Allele Frequency Net Database (https://www.allelefrequencies.net/). The chosen HLA-ABC alleles are listed in FIG. 4. The coding sequences of the chosen HLA class I alleles were obtained from the IDP-IMGT/HLA database (https://www.ebi.ac.uk/ipd/imgt/hla/) and were aligned using Geneious Prime. Those potential regions were analyzed using the Genetic Perturbation Platform with default parameters to predict efficient shRNAs, and the top-ranked shRNAs were selected for further investigation.

### NK Cell Isolation, CIML Stimulation, Expansion, and Resting

Whole blood samples from healthy donors were provided Dana-Farber Cancer Institute at Harvard Medical School. Peripheral blood mononuclear cells (PBMCs) were first isolated using Ficoll-Paque density gradient media (Cytiva) with SepMate™ PBMC isolation tubes (STEMCELL) according to the instructions. The isolated PBMCs were either used for NK cell or T cell isolation immediately or frozen down to liquid nitrogen for later use. All the primary NK cells used in the present study are cytokine-induced memory-like (CIML) NK cells as described previously (Romee et al., 2012). NK cells were isolated using the human NK cell isolation kit from Miltenyi Biotec according to the manufacturer's instructions. NK cells were subjected to CIML stimulation immediately. Briefly, NK cells were adjusted to 3-4 million per ml in NK MACS medium supplemented with 10% human AB serum and 1% penicillin/streptomycin (NK complete media), and 3 ml of NK cells were seeded to one well of a 6-well plate. For CIML stimulation, recombinant human IL-12 (Miltenyi), IL-15 (Miltenyi), and IL-18 (Miltenyi) were added to NK complete media to concentrations of 10 ng/ml, 50 ng/ml, and 50 ng/ml, respectively. The NK cells were incubated at 37° C., 5% CO2 for 18-24 hours. Successfully CIML-stimulated NK cells will form clusters and attain sticky/branched morphology. When NK cells reach this state, the CIML stimulation media should be completely replaced the expansion media (fresh NK complete media supplemented with 500 IU/ml of IL-2 (Miltenyi) and 5 ng/ml of IL-15 (Miltenyi). NK cells can be expanded in the expansion media for about two weeks. After the NK cell expansion is completed, the expansion media should be replaced with fresh NK complete media supplemented with 1 ng/ml of IL-15 for two to three days before any functional assays.

### Production of Lentiviral Vectors and Transduction of NK Cells

Lentiviruses with baboon endogenous retroviral envelope (BaEV) (Dong et al., 2022) and titrated on Jarkat T cells were pseudotyped. The titrated lentiviral vectors were used to transduce NK cells derived from PBMCs of healthy donors at a multiplicity of infection (MOI) of 10. Briefly, suspension 293T cells were adjusted to 4 M/ml and transfected with the transfer plasmid encoding the gene of interest, the envelop plasmid encoding the BaEV (derived from pMD2.G by replacing the VSVG with BaEV encoding sequence), the packaging plasmid (pCMV-dR8.91), and the pAdVAntage plasmid, complexed with polyethyleneimine (PEI) at an optimal mass ratio. The transfected 293T cells were pelleted and resuspended in fresh media at 15 hours post-transfection. The supernatant 1 containing the lentiviral vectors was collected by pelleting the cells 24 hours later. The cells were resuspended in fresh media and cultured for another 24 hours. The supernatant 2 was collected by spinning down the cells. Supernatant 1 and supernatant 2 were combined, and centrifuged at 3000 rpm, 4° C., for 10 min to remove the cell debris. The cleared supernatant was then filtered through the 0.45 μm filters and the lentiviral vector was concentrated by centrifugation at 10000 g, 4° C., for 20 hours. The virus pellet was resuspended in PBS, aliquoted, and frozen down at −80° C. for later use. After that, the lentiviral vectors were titrated on Jurkat T cells. 0.5 million Jurkat T cells were transduced with a serial amount of virus with 8 μg/ml polybrene (Sigma-Aldrich). The well that gave around 10-20% positive cells was used for calculating the viral titer with the formula: titer (TU/ml)=(500000*% positive)/virus volume.

The CIML-stimulated NK cells were transduced with the lentiviral vector in the presence of 10 μg/ml of Vectofusion-1 (Miltenyi) in the NK expansion media (described above) at MOI of 10. After adding the virus, the cells were centrifuged at 1000 g, 37° C. for 1.5 hours. The cells were then put back in the incubator and cultured for 3 days prior to the examination of the expression of the protein of interest.

### In Vitro NK Cell Functional and Killing Assays

CIML-stimulated and expanded NK cells were rested for at least 3 days prior to any functional and killing assays. For the killing of suspension target cells, the cells were pre-labeled with CTV and seeded at 2×104 in the v-bottom 96-well plate, and the NK cells were added according to the E:T ratios. For the killing of adherent target cells (OVCAR8), the cells were pre-labeled with CTV and seeded at 2×104 in the flat-bottom 96-well plate and incubated overnight to allow cell attachment to the plate. The NK cells were added according to the E:T ratios and co-cultured with target cells overnight. After co-culture, the supernatant was transferred to a v-bottom plate, and the attached cells in the flat-bottom 96-well plate were trypsinized and added to the corresponding wells in the v-bottom plate. All the NK cell-killing assay was conducted in the presence of 1 ng/ml of IL-15. CD107a and IFNγ expression in NK cells was measured by staining the cells with anti-CD56, anti-CD107a, and anti-IFNγ antibodies and determined by flow cytometry. For the lysis of target cells, the cells were stained with Zombie NIR (BioLegend), and the precision counting beads (BioLegend) were added before flow cytometry. The CTV positive and Zombie NIR negative cells (live target cells) were counted using the formula:

$$Absolute cell number = \frac{cell count}{beads count} \times ( added beads volume \times beads concentration )$$

The percentage of target cell lysis was calculated by (total cell number−live cell number)/total cell number.

### In Vitro CD8+ T Cell Killing Assay

First feeder allogeneic NK cells were prepared by culturing them in RPMI-1640 containing 10 μg/ml mitomycin C at 37° C. with 5% CO2 for 3 hours. The cells were then centrifuged and washed with PBS three times. After that, freshly isolated CD8+ T cells were primed with mitomycin C-pretreated allogeneic NK cells in the presence of 500 ng/ml IL-2 for 7 days. Primed CD8+ T cells were sorted (Miltenyi) by negative selection for the subsequent killing assay. The primed CD8+ T cells were cocultured with CTV prelabeled NK cells (from the same donor as the priming NK cells) with either normal or reduced HLA-ABC expression (due to knockdown by shRNA #1 or #13), or with or without PD-L1 and SCE overexpression, at indicated E:T ratios in the v-bottom 96-well plate overnight. After coculture, the cells were centrifuged and stained with Zombie NIR, and the precision counting beads were added before flow cytometry. The CTV-positive and Zombie NIR negative cells were the live NK cells. The lysis of target cells by CD8 T cells was calculated as described above.

### Mixed Lymphocyte Reaction

Freshly isolated CD3+ T cells were cocultured with autologous or HLA mismatched NK cells with or without PD-L1 overexpression in a v-bottom 96-well plate at a ratio of 1:1. The cell mixture was mixed by pipetting and centrifuged at 1000 rpm for 1 min. Twelve hours later, the cells were centrifuged and stained with anti-CD3, anti-CD25, and anti-CD69 antibodies and analyzed by flow cytometry. The CD3+CD25+ and CD3+CD69+ cells are the CD25− and CD69− expressing T cells, respectively. In a separate experiment, CTV prelabled CD3+ T cells were cocultured with autologous or HLA mismatched allogeneic NK cells with or without PD-L1 overexpression in a v-bottom 96-well plate at a ratio of 1:1 for 5 days, and the proliferation of CD3+ T cell was determined by dilution of CTV using flow cytometry.

### Mouse Studies

All mice were sex-matched and age-matched (8-10-week-old) into groups and irradiated with 100 cGy two days before infusion of human cells. Before proceeding to any mouse experiment, the successful NK cell transduction was first confirmed. For instance, NK cell transduced with a lentivector expressing shRNA #1+PD-L1 showed reduced surface HLA-ABC and high level of surface PD-L1, and NK cell transduced with a lentivector 879 expressing shRNA #1+CD19CAR+PD880 L1 showed reduced surface HLA-ABC and expression of CD19 CAR and HLA-E. All transduced NK cells were used within two weeks at the time of infusion.

For assaying in vivo allogeneic responses, 10 million NK cells with HLA-ABC knockdown but overexpression of PD-L1 or SCE were mixed with 10 million PBMCs from an HLA class I mismatched donor, infused into NSG MHC I/II DKO mice through intravenous (i.v.) injections. Before infusion, the percentages of NK cells (donor 1 transduced NK cell, donor 1 untransduced NK cell, and donor 2 NK cell) and donor 2 T cell were characterized in detail by staining the cells with anti-hCD45-FITC, anti-CD3-BUV395, anti-CD56-PE-Cy7, anti-HLA-ABC-PE, anti-HLA-A2-PerCP-Cy5.5, anti-PD-L1-APC (or anti-HLA-E-APC when SCE overexpressed cells were infused), anti-CD69-BV711, anti-anti-CD107a-APC-Cy7 and evaluated by flow cytometry. The antibody panel was designed using FluoroFinder and evaluated. Peripheral blood from mice was obtained on day 7, day 14, and day 21, by facial bleeding into an EDTA-coated tube. Red blood cells were lysed with 4 ml (10× the blood volume) ACK buffer for 10 mins on the ice. Cells were then washed twice with FACS buffer (PBS+1% BSA) and then stained with the above antibody panel. Mice were euthanized on day 21, lung, liver, spleen, and bone marrow were collected, and leucocytes were isolated from the organs as described previously. In brief, the tissues (lung, liver, and spleen) were diced using surgical scissors on a 10-cm sterile cell culture plate and resuspended in 4 ml RPMI-1640. The diced tissues were further homogenized with gentleMACS Tissue Dissociator. Leucocytes were isolated using the Percoll density separation method. The bone marrow cells were aspirated using a 28 G insulin needle, lysed with ACK buffer, and filtered through a 40 μm cell strainer to acquire a single-cell suspension. After separation, the cells from the tissues were stained with the above antibodies. All the samples were analyzed on a FACS Symphony A3 instrument.

For tumor killing experiment, xenograft models were established by intravenous injection of 0.5×106 Raji-luciferase cells into NSG MHC I/II DKO mice. Two days later, 4 million allogeneic CAR NK cells and 4 million PBMCs from a different donor were infused into mice through intravenous injection. Tumor burden was assessed on day 7, day 14, and day 21, using the IVIS Lumina Series III (Perkin Elmer). BLI intensity was analyzed by Aura imaging analysis software. Blood was collected on day 14, and blood, lung, liver, spleen, and bone marrow were collected when the mice met the prespecified endpoints according to the CAC protocols. The blood and tissues were processed and stained with antibodies, as described above.

### Flow Cytometry

Flow cytometry was performed on a FACS Symphony A3 instrument and analyzed using FlowJo software. Cell sorting was performed using a FACS Aria 3 sorter.

### Rna-Seq, Mapping, and Analysis

NK cells isolated from 4 unrelated donors were transduced with lentivectors expressing shRNA #1+MLSN CAR, shRNA #1+MLSN CAR+PD-L1, and shRNA #1+MLSN CAR+SCE, respectively. Untransduced NK cells from the same donors were used as controls. NK cells were labeled with CTV cultured in media alone or cocultured with OVCAR8 cells for 18 hours in triplicates. The cocultured NK cells were purified by FACS sorting the CTV+ cells, and NK cells without coculture were obtained by pipetting from the wells. For RNA-seq, around 0.5×106 live NK cells for each sample were processed. RNA extraction and cDNA libraries were performed using the NEBNext Ultra II RNA Library Prep Kit for Illumina (NEB #E7770L) per the manufacturer's instructions. Libraries were sequenced using the Illumina NovaSeq SP 100-nt kit. RNA-seq reads were aligned to the human genome with Salmon (v0.14.1) using ensembl Homo sapiens GRCh38 (release 112) transcript annotations. The resulting counts were analyzed in R (v4.3.2) using tximport (v1.30.0) and DESeq2 (v 1.42.1) for differential expression analysis. The Wald test and Bonferroni post-hoc correction were used to identify differentially expressed genes. Log fold change (LFC) shrinkage was performed on differentially expressed genes with apeglm LFC shrinkage algorithm53. Gene set enrichment analysis (GSEA) was performed using fgsea (v 1.29.1), and the p-values were calculated with weighted Kolmogorov-Smirnov statistic tests and adjusted using the Benjamini-Hochberg correction. Gene sets were curated from the msigDB (v 7.5.1) database. Data visualization was done with ggplot2 (v3.5.0).

### Statistical Analysis

The data were analyzed statistically using Prism Version 8 or SPSS Statistics Package 25 (SPSS Inc. Chicago, USA). One-way or two-way ANOVA with Tukey's multiple comparisons was performed between the indicated groups to determine the statistical difference. For the survival of mice, the data were analyzed using Kaplan-Meier method incorporated in Prism.

### Results

Identification of a Potent shRNA for Specific Knockdown of HLA-ABC without Affecting HLA-E Expression

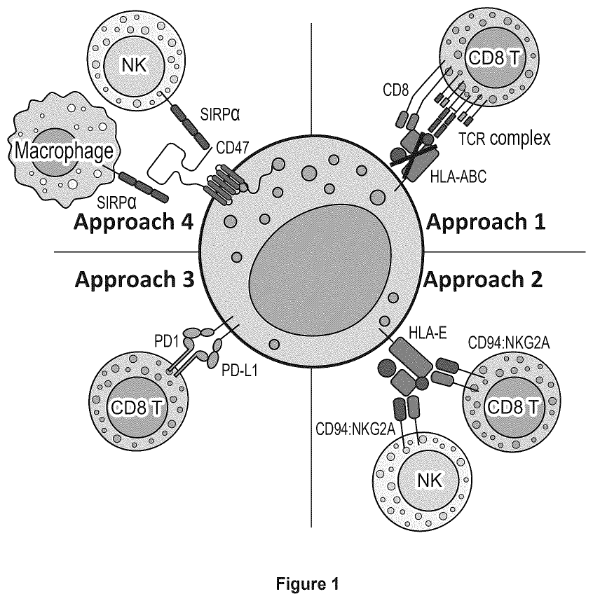

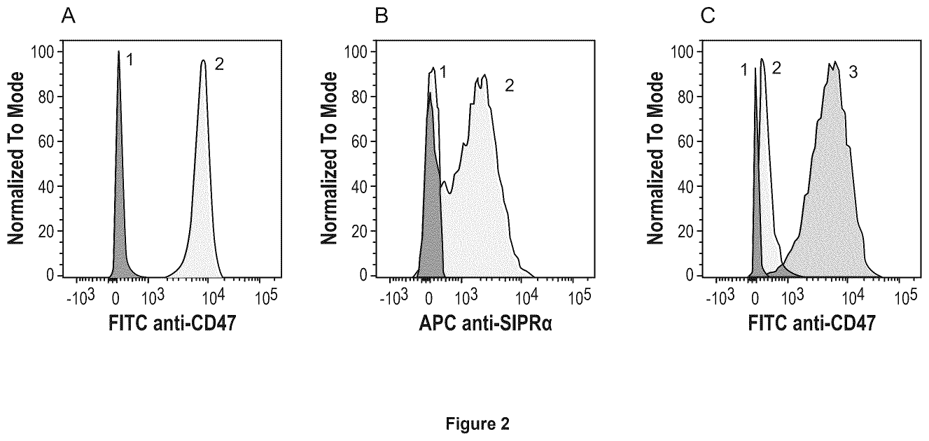

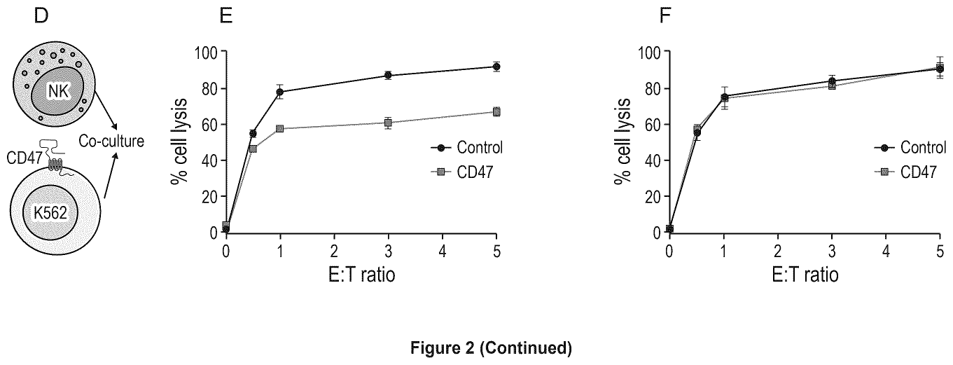

To develop allogeneic CAR-NK cells that could escape rejection by host CD8+ T cells, NK cells and macrophages, a tiered approach was used (FIG. 1). An shRNA was developed to knockdown HLA-ABC with the goal of suppressing T cell-mediated rejection and integrating the shRNA into the same CAR lentivector, for one-step construction of allogeneic CAR NK cells. Expression of HLA-E and/or PD-L1 was evaluated to further suppress rejection of allogeneic NK cells by host CD8+ T and NK cells. CD47-SIRPα was evaluated for interaction to suppress macrophage phagocytosis of allogeneic NK cells (FIG. 1). Interestingly, human peripheral blood NK cells expressed a high level of CD47 and the majority of them also expressed SIRP α (FIG. 2a-b). Expression of CD47 in K562 cells, a human lymphoblast cell line with no expression of HLA-ABC and HLA-E, significantly inhibited killing by human NK cells (FIG. 2c-d), suggesting a significant role of CD47-SIRPα interaction in inhibiting cytotoxicity of NK cells. Blocking the CD47-SIRPα interaction with an anti-CD47 antibody abolished CD47-mediated inhibition (FIG. 2e-f). Thus, CD47-SIPRα interaction also likely plays an important role in inhibiting cytotoxicity of NK cells. Because CD47 and SIRP□ are highly expressed by NK cells, no further manipulation of this interaction was explored.

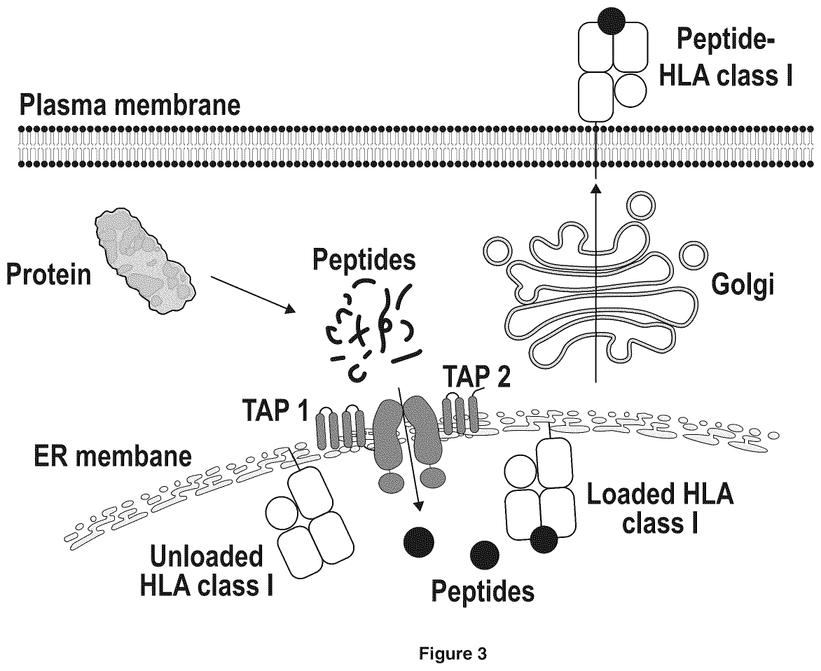

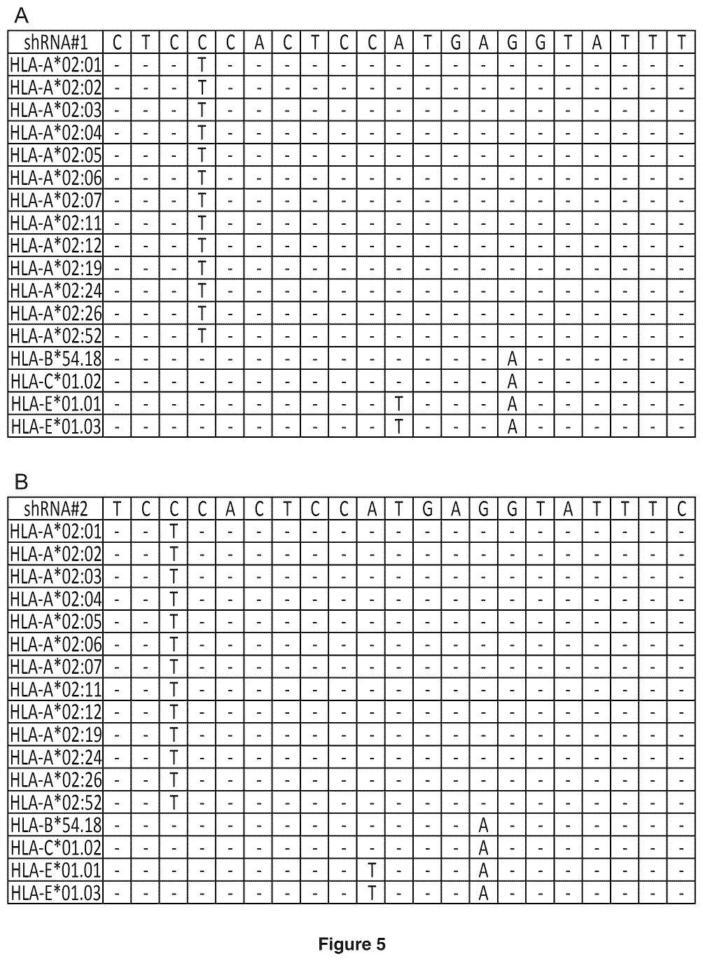

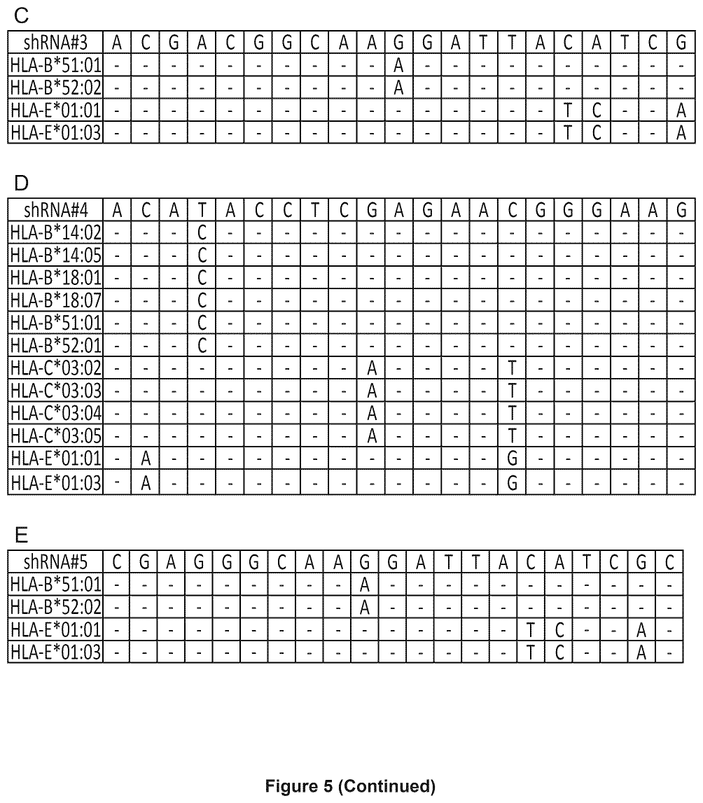

Multiple genes were targeted for knock down HLA-ABC by RNA interference, including the HLA126 ABC heavy chain, B2M, and TAP1 and TAP2 (FIG. 3). To specifically knock down HLA-ABC without affecting HLA-E, the coding sequences of the most prevalent classical HLA class I alleles across global populations were aligned (FIG. 4), including 38 HLA-A alleles, 56 HLA129 B alleles, 25 HLA-C alleles, as well as 2 nonclassical HLA-E alleles (E*01:01 and E*01:03), and designed shRNAs were designed that had no more than one mismatch with HLA-ABC alleles but had at least two mismatches with HLA-E alleles (Table 2 and FIG. 5a-e).

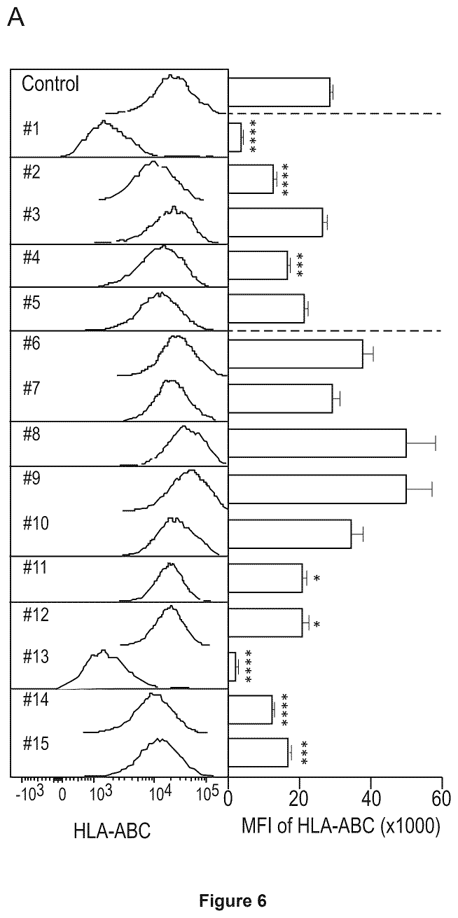

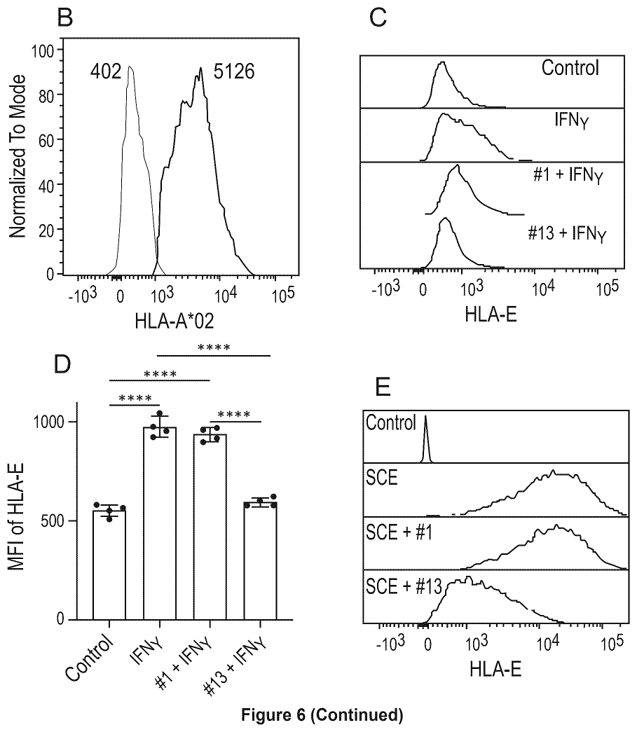

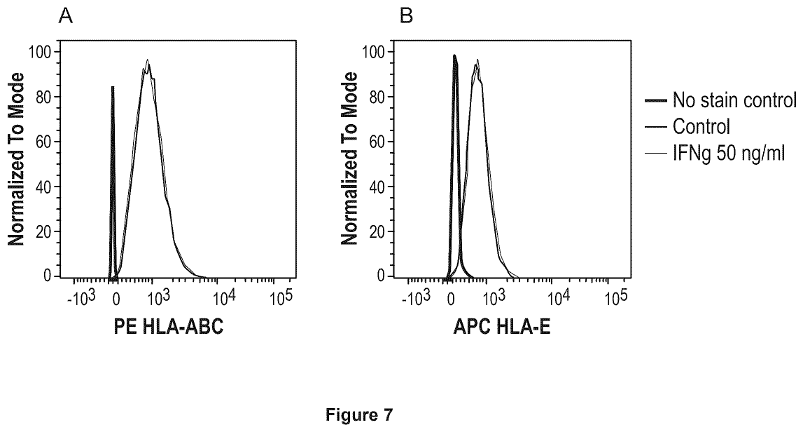

Lentivectors were constructed expressing shRNAs under the control of the U6 promoter, transduced human Jurkat T cells, and assayed HLA-ABC expression by flow cytometry 48 hours later. Among the 15 shRNAs tested, shRNA #1 and #2 (targeting HLA-ABC heavy chain), #13 and #14 (targeting B2M) were most potent in inhibiting surface HLA-ABC expression (FIG. 6a). These four shRNAs also significantly knocked down surface HLA-ABC expression in NK cells from four donors (FIG. 6a-b). Because shRNA #1, #2, #13, and #14 were the most potent, they were used in the subsequent studies.

shRNA #1 had one nucleotide mismatch with the prevalent HLA-A*02 alleles and two nucleotide mismatches with HLA-E alleles (FIG. 5). To investigate if shRNA #1 was still effective to knock down HLA-A*02 expression, NK cells were isolated from HLA-A*02+ donors, transduced with lentivector expressing shRNA #1, and assayed HLA-A*02 expression by flow cytometry. Compared to untransduced NK cells, HLA-A*02 expression was reduced by ˜13-fold in transduced NK cells (FIG. 6c), demonstrating effective knockdown of HLA-A*02 expression by shRNA #1 despite one nucleotide mismatch.

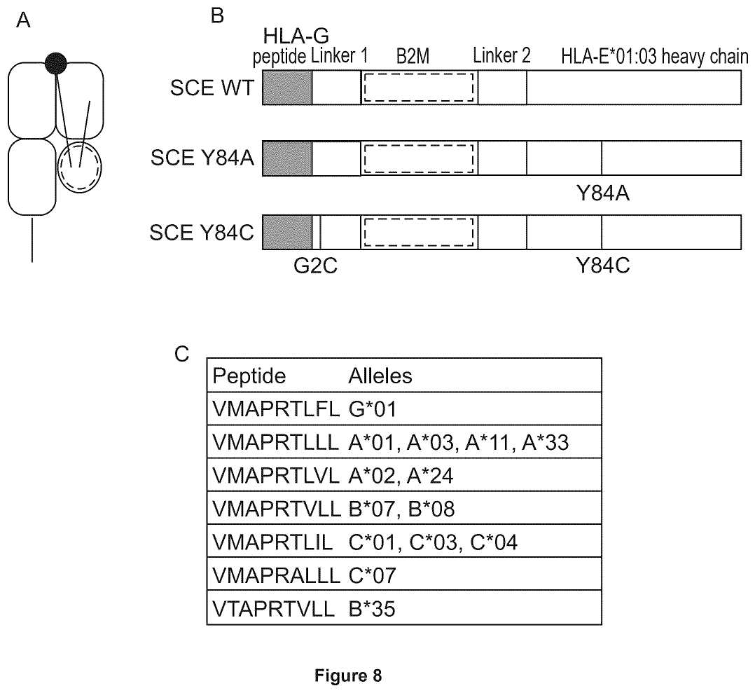

It was also determined whether shRNA #1 interferes with HLA-147 expression. IFNγ treatment of Jurkat T cells significantly induced HLA-E expression as indicated by an increase in MFI from 510 to 980 (FIG. 6d-e). Expression of shRNA #1 in Jurkat T cells did not significantly reduce the IFNγ-induced surface HLA-E expression, whereas shRNA #13, which targets B2M, completely inhibited the IFNγ-induced surface HLA-E expression. Alternatively, single chain HLA-E was constructed (SCE) where HLA-E*01:03 heavy chain, B2M, and the peptide from the HLA-G leader sequence were connected by two linkers into a single polypeptide (see FIG. 8a-b). Transduction with the lentivector expressing SCE into K562 cells led to a high level of HLA-E expression (FIG. 6e), which was not reduced by shRNA #1 but reduced by shRNA #13. These results show that shRNA #1, #2, #13, and #14 could potently knock down surface HLA-ABC expression and shRNA #1 specifically knocked down surface HLA-ABC expression without affecting HLA-E expression.

### HLA-ABC-Reduced Allogeneic NK Cells are Resistant to Killing by Host CD8+ T Cells

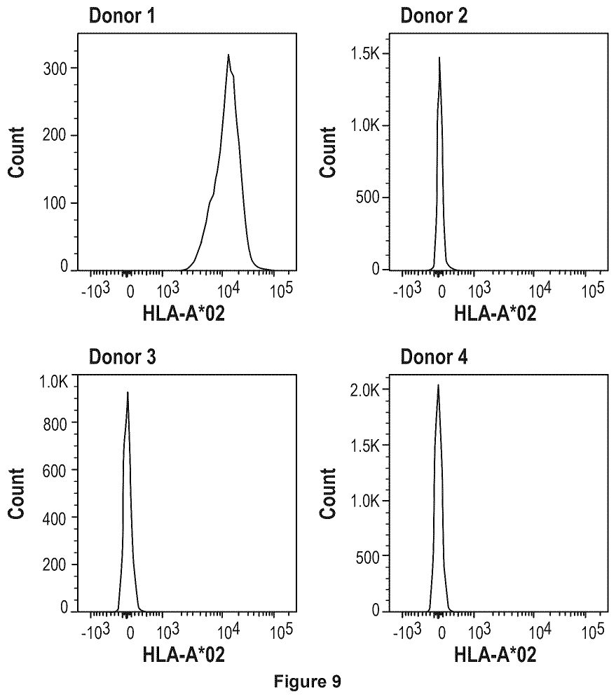

Whether surface HLA-ABC-reduced allogeneic NK cells are resistant to killing by host CD8+ T cells and NK cells was investigated. NK cells and CD8+ T cells were purified from peripheral blood mononuclear cells (PBMCs) from four unrelated healthy donors (referred to as donor 1, donor 2, donor 3 and donor 4). Donor 1 was HLA-A*02:01-positive and the other three donors were HLA165 A*02:01-negative (FIG. 9). Purified NK cells from the four donors were transduced with lentivector expressing shRNA #1, #2, #13, or #14, and HLA-ABC-reduced NK cells were purified by cell sorting (based on reduced HLA-ABC expression), expanded and used as targets for allogenic CD8+ T cell and NK cell killing.

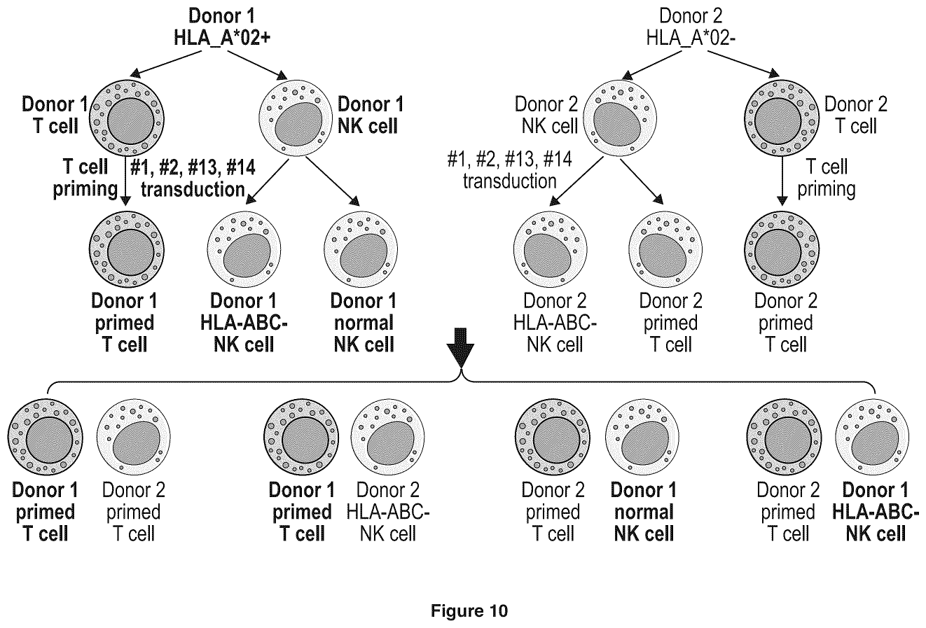

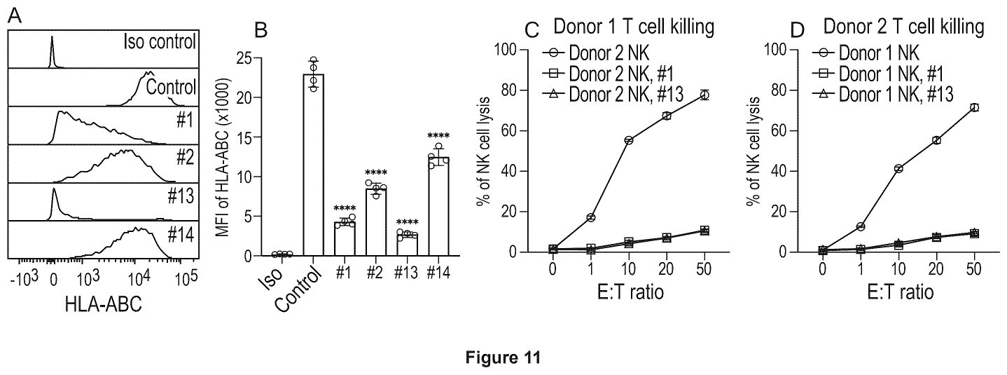

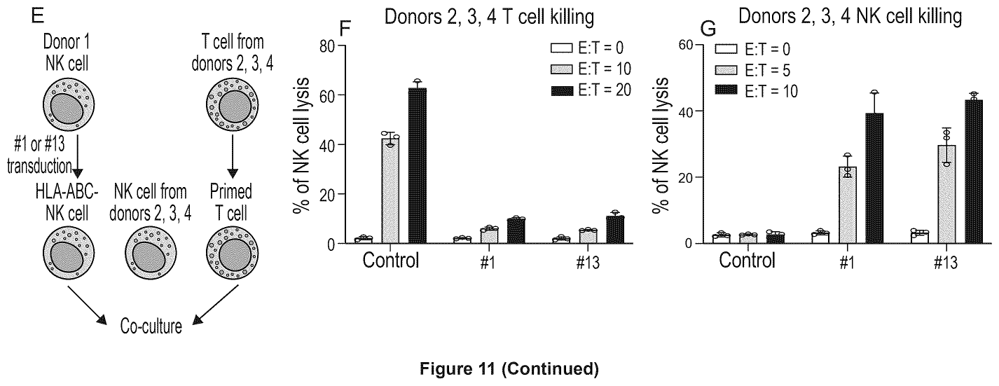

CD8+ T cells from donor 1 were first primed with mitomycin C pretreated NK cells from donor 2 (with normal HLA-ABC expression) in the presence of IL-2 for 7 days (FIG. 10). The primed CD8+ T cells were then cocultured with prelabeled NK cells from donor 2 with either normal or reduced HLA-ABC expression overnight at E:T ratios of 0, 1, 10, 20 and 50, and the lysis of donor 2 NK cells was determined by flow cytometry. As shown in FIG. 11c, donor 1 CD8+ T cells killed donor 2 NK cells with normal HLA-ABC expression in a dosage-dependent manner, reaching 80% killing at E:T ratio of 50. In contrast, donor 1 CD8+ T cell killing of donor 2 NK cells with reduced HLA-ABC expression was reduced to ˜10% at E:T ratio of 50. In a reciprocal experiment, donor 2 CD8+ T cells efficiently killed donor 1 NK cells with normal HLA-ABC expression, but the killing was reduced following HLA-ABC knockdown on donor 1 NK cells (FIG. 11d).

In a separate experiment, CD8+ T cells from donors 2, 3 and 4 were primed with mitomycin C pretreated donor 1 NK cells in the presence of IL-2 for 7 days. The primed CD8+ T cells were then cocultured with prelabeled donor 1 NK cells with either normal or reduced HLA-ABC expression overnight at E:T ratios of 0, 10 and 20, and the lysis of donor 1 NK cells was determined (FIG. 11e). Donor 1 NK cells with normal HLA-ABC expression were lysed in a dosage-dependent manner by CD8+ T cells from all three donors, reaching 40% and 60% at E:T ratios of 10 and 20, respectively (FIG. 11f). In contrast, lysis of donor 1 NK cells with reduced HLA-ABC expression was reduced to ˜10% at E:T ratio of 20. Thus, knockdown of HLA-ABC expression in allogeneic NK cells by shRNA makes them resistant to host CD8+ T cell killing in vitro.

To test whether surface HLA-ABC-reduced allogeneic NK cells are sensitive to host NK cell killing, 1 cells from donors 2-4 were cocultured with prelabeled NK cells from donor 1 with either normal or reduced HLA-ABC expression at E:T ratios of 0, 5 and 10 (FIG. 11e). Because NK cells express high levels of both CD47 and SIRPα, whose interaction blocks NK cell killing (FIG. 2), anti-CD47 antibody and human FcR blocking antibodies were added into the coculture to inhibit CD47-SIRPα axis and antibody-dependent cellular cytotoxicity (ADCC). Donor 1 NK cells with normal HLA-ABC expression were not killed appreciably, whereas donor 1 NK cells with reduced HLA-ABC expression were killed in a dose-dependent manner, reaching ˜40% at E:T ratio of 10 (FIG. 11g). Together, these results show that the knockdown of HLA-ABC expression in allogeneic human NK cells by both shRNA #1 and #13 makes them resistant to CD8+ T cell killing but sensitizes them to NK cell killing when CD47-SIRP α interaction is blocked.

### Exogenous Expression of PD-L1 in NK Cells Moderately Inhibits Allogeneic T Cell Responses

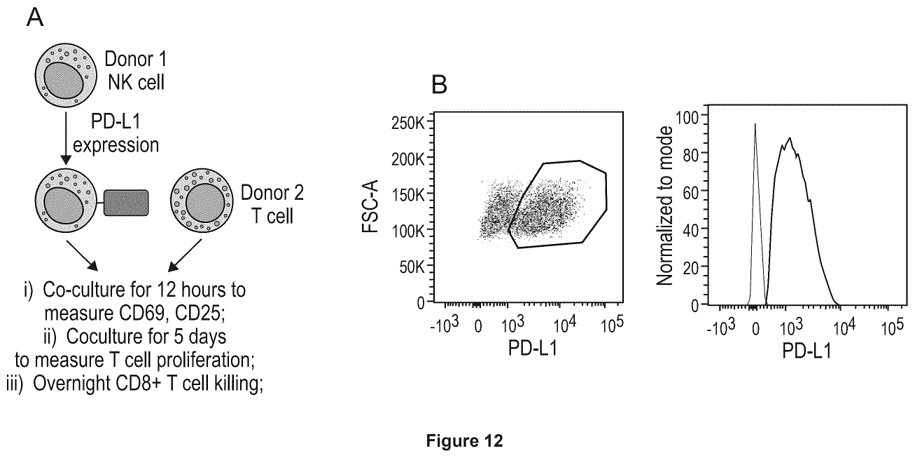

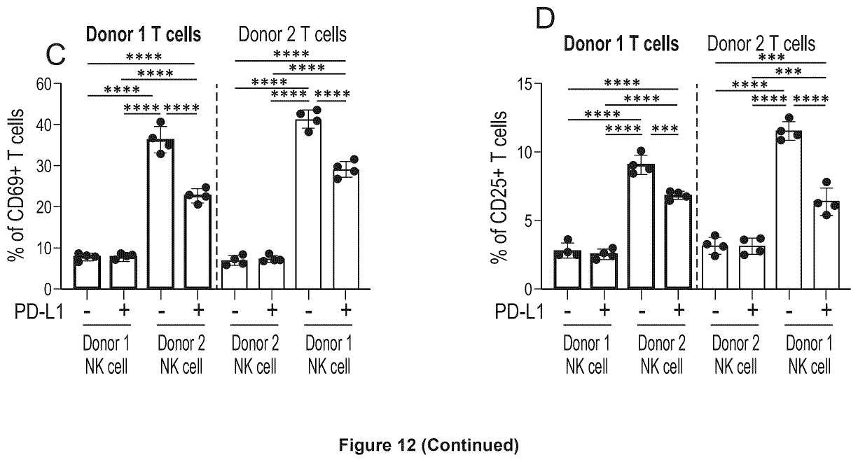

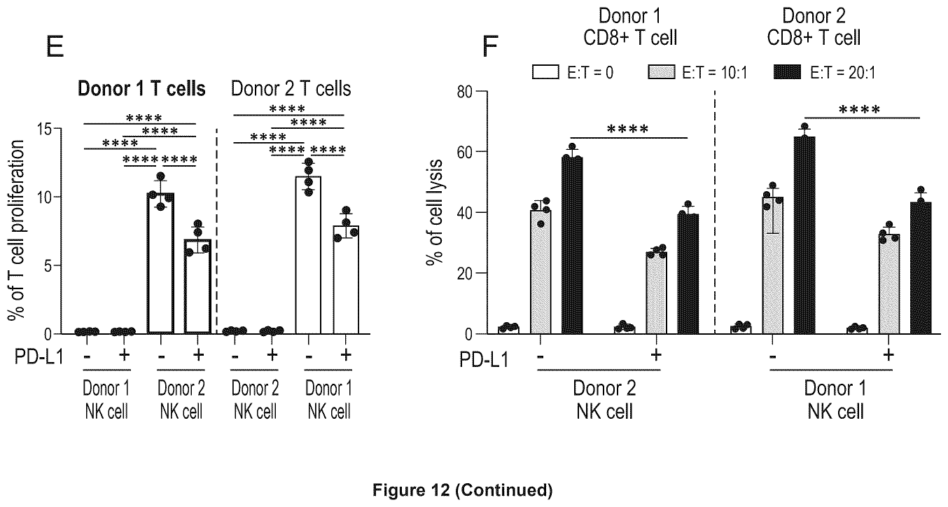

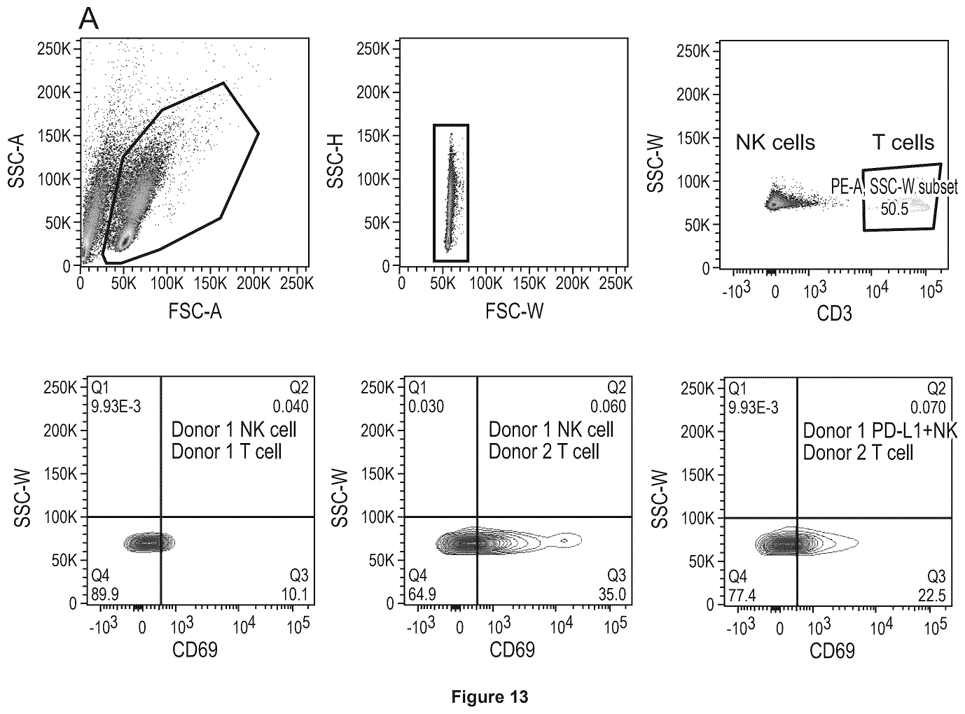

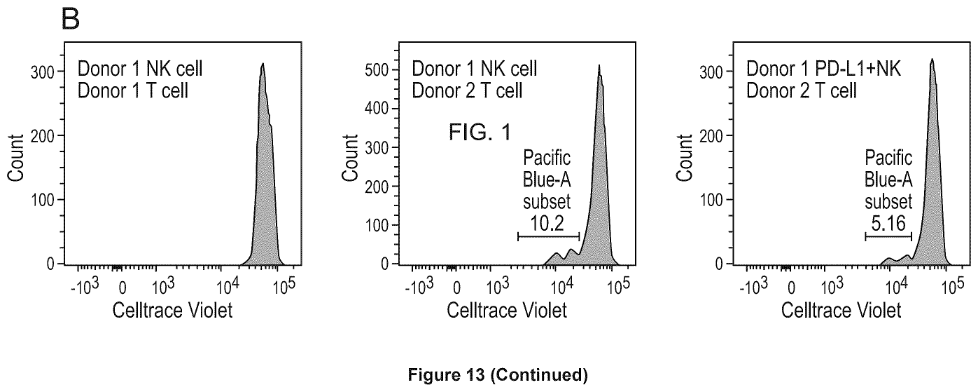

To investigate the effect of PD-1-PD-L1 interaction in the control of T cell responses to allogeneic NK cells, reciprocal experiments were performed with T cells and NK cells from donors 1 and 2 (FIG. 12a). NK cells were transduced with lentivector expressing PD-L1 and PD-L1+NK cells were sorted and expanded (FIG. 12b). CD3+ T cells from the same two donors were cocultured with autologous or allogeneic NK cells with or without PD-L1 expression at a ratio of 1:1. CD69 and CD25 expression by T cells was measured 12 hours post-coculture (FIG. 12a). Fewer than 10% of T cells expressed CD69 when they were cocultured with autologous NK cells with or without exogenous PD-L1 expression (FIG. 12c and FIG. 13a). Between 35%-40% of T cells were induced to express CD69 when they were cocultured with allogeneic 247 NK cells without exogenous PD-L1 expression. The percentage of CD69+ T cells was reduced by 20-30% when cocultured with allogeneic NK cells with exogenous PD-L1 expression (FIG. 12c). Similarly, <3% T cells expressed CD25 when cocultured with autologous NK cells, whereas 9%-12% T cells expressed CD25 when cocultured with allogeneic NK cells without exogenous PD-L1 expression and the percentage was decreased to ˜7% when cocultured with allogeneic NK cells with exogenous PD253 L1 expression (FIG. 12d). T cells did not proliferate significantly when cocultured with autologous NK cells with or without exogenous PD-L1 expression, whereas 10-12% T cells underwent proliferation when cocultured with allogeneic NK cells without PD-L1 expression and the percentage was reduced to ˜7% when cocultured with allogeneic NK with PD-L1 expression (FIG. 12e and FIG. 13b). When primed CD8+ T cells were cocultured with allogeneic NK cells with or without exogenous PD-L1 expression at the E:T ratios of 10 and 20, ˜40% and ˜60% of untransduced allogeneic NK cells were lysed, respectively, whereas lysis of allogeneic NK cells expressing PD-L1 was reduced to ˜30% and ˜40% at E:T ratios of 10 and 20, respectively (FIG. 12f). Together, these results show that exogenous expression of PD-L1 on allogeneic NK cells partially inhibits host T cell responses.

### Exogenous Expression of Single-Chain HLA-E Inhibits Killing of Allogeneic NK Cells by Host NK Cells

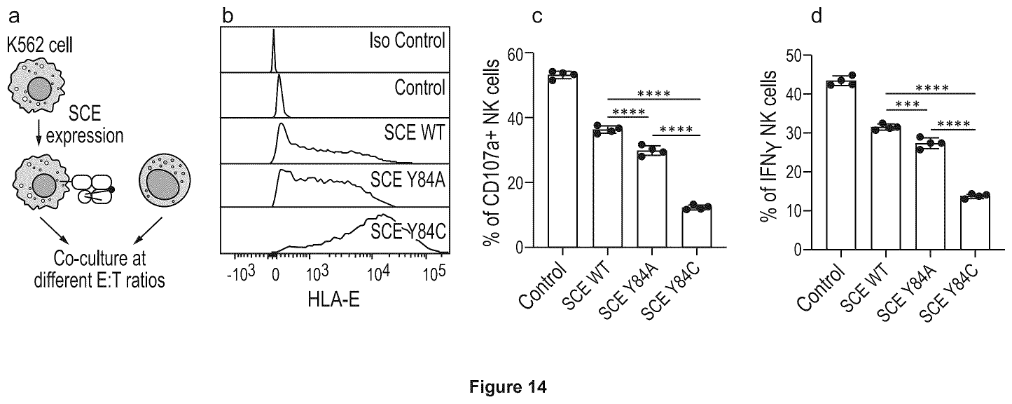

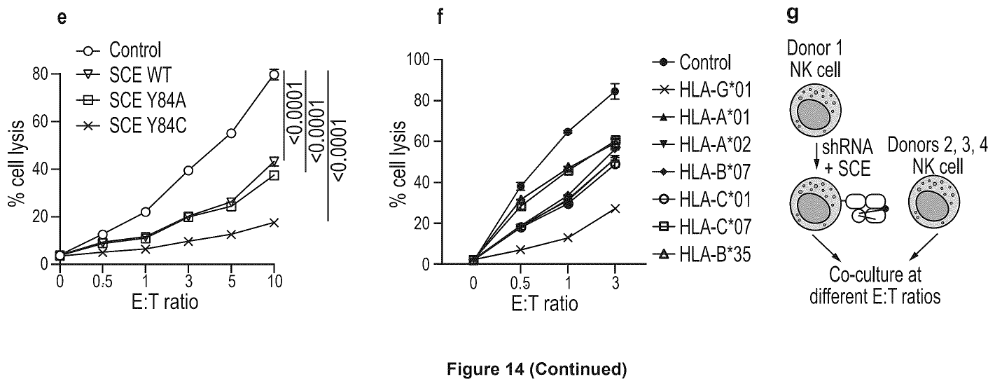

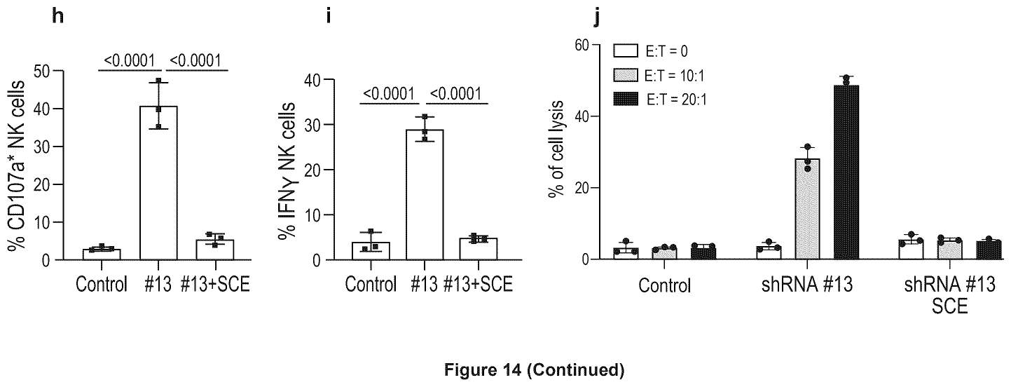

Single-chain HLA class I incorporates HLA class I heavy chain, B2M, and the presented peptide into a single polypeptide with two linkers (FIG. 8a), with mutation of tyrosine 84 in the wildtype (WT) heavy chain to either alanine (Y84A) or to cysteine (Y84C) for better fitting of the peptide into the peptide-binding groove (FIG. 8b). The same approach was used to express three forms of single-chain HLA-E (SCE) consisting of the peptide derived from the HLA-G leader sequence (VMAPRTLFL, SEQ ID NO: 31), B2M, and HLA-E*01:03 heavy chain (FIG. 14a-b). Lentivectors expressing the SCE variants were used to transduce K562 cells; all three SCE variants supported surface HLA-E expression, with SCE Y84C showing the highest level of expression (FIG. 14a-b) when transduced at the same MOI.

To evaluate the functionality of SCE variants, purified human NK cells were cocultured with K562 cells with or without SCE expression (FIG. 14a). NK cell degranulation (CD107a), IFNγ expression and lysis of K562 cells were measured 4 hours post-coculture. Approximately 55% of NK cells were induced to express CD107a following coculture with the parental K562 cells (FIG. 14c). This percentage was significantly reduced to ˜35%, ˜30% and ˜10% by K562 cells expressing SCE WT, Y84A and Y84C variants, respectively. Similarly, the percentages of IFNγ+ NK cells were significantly reduced from ˜40% following coculture with the parental K562 cells to ˜15-30% following coculture with K562 cells expressing the SCE variants, with the SCE Y84C showing the most potent inhibition (FIG. 14d). In addition, human NK cells effectively killed the parental K562 cells with increasing E:T ratios (FIG. 14e), whereas killing of K562 cells was significantly inhibited by expression of the SCE variants, especially SCE Y84C (80% vs 20% lysis at E:T ratio of 10).

HLA-E presents peptides derived from the leader sequences of the heavy chains of HLA-ABC and HLA-G. Because amino acid sequence variations in the leader sequences, the peptide from HLA-G and 6 peptides from 13 prevalent HLA-ABC alleles were tested (FIG. 14). K562 cells were transduced with lentivectors expressing SCE Y84C presenting these different peptides, cocultured with human NK cells for 4 hours at the E:T ratios of 0.5, 1 and 3. Human NK cells efficiently lysed the parental K562 cells, reaching ˜85% at E:T ratio of 3 (FIG. 14f). Expression of SCE Y84C presenting different peptides all significantly inhibited lysis of the transduced K562 cells, but SCE presenting the HLA-G peptide was the most potent in inhibiting NK cell lysis of K562 cells. Thus, SCE Y84C presenting the HLA-G peptide was used in all subsequent studies.

TABLE 3
Exemplary Leader Peptide Sequences

| Peptide | Alleles | SEQ ID NO: |
|---|---|---|
| VMAPRTLFL | G*01 | 31 |
| VMAPRTLLL | A*01, A*03, A*11, A*33 | 32 |
| VMAPRTLVL | A*02, A*24 | 33 |
| VMAPRTVLL | B*07, B*08 | 34 |
| VMAPRTLIL | C*01, C*03, C*04 | 35 |
| VMAPRALLL | C*07 | 36 |
| VTAPRTVLL | B*35 | 37 |

Whether SCE Y84C expression could protect HLA-ABC-reduced allogeneic NK cells from killing by the host NK cells was tested. shRNA #13 was used to knock down HLA-ABC expression because shRNA #13 was more potent than shRNA #1 in order to enable a more stringent killing assay. NK cells from donor 1 were transduced with lentivector expressing shRNA #13 or shRNA #13 plus SCE Y84C, and the transduced NK cells were purified and expanded (FIG. 14g). NK cells from donors 2-4 were cocultured with donor 1 NK cells with normal or reduced HLA-ABC expression, or reduced HLA-ABC but exogenous SCE Y84C expression at E:T ratios of 0, 5 and 6 hours (in the presence of anti-CD47 and Fc blocker antibodies). NK cells were not induced to express CD107a and IFNγ following coculture with allogeneic NK cells with normal HLA-ABC expression, but were significantly induced to express CD107a and IFNγ following coculture with HLA-ABC-reduced NK cells (FIG. 14h-i). Exogenous expression of SCE Y84C in HLA-ABC330 reduced NK cells almost completely inhibited the induction of CD107a and IFNγ following coculture (FIG. 3h-i). Similarly, NK cells did not kill allogeneic NK cells with normal HLA-ABC expression, but killed HLA-ABC-reduced NK cells, which was completely inhibited by exogenous expression of SCE Y84C (FIG. 14j). These results show that exogenous expression of SCE Y84C inhibits host NK cell killing of allogeneic NK cells with reduced HLA-ABC expression.

### Combination of HLA-ABC Knockdown and PD-L1 or SCE Expression Confers Resistance to Allogeneic Immune Cell Killing In Vitro

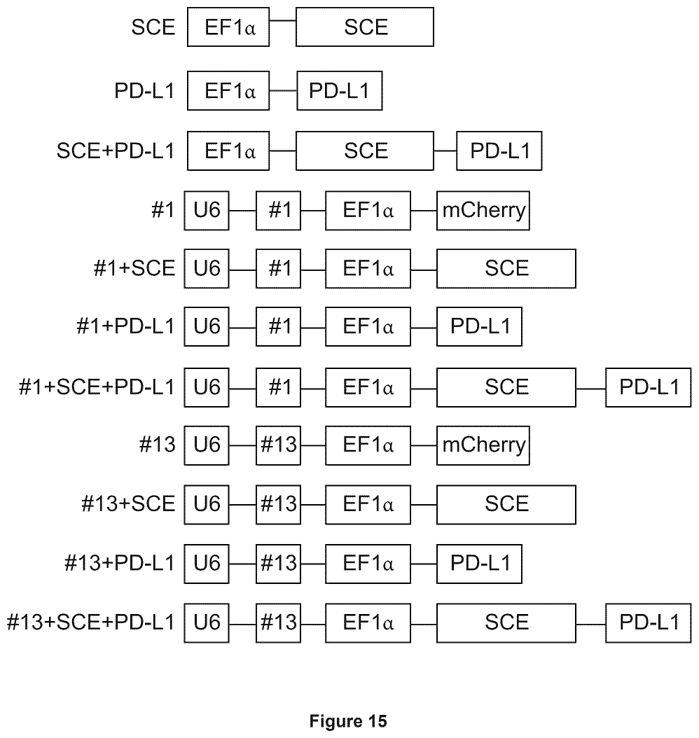

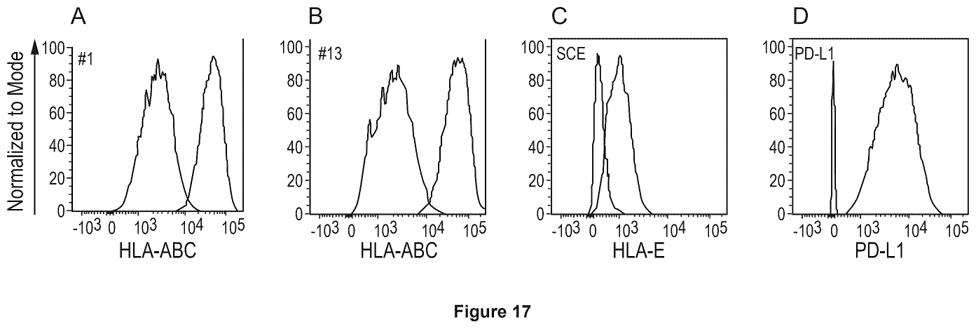

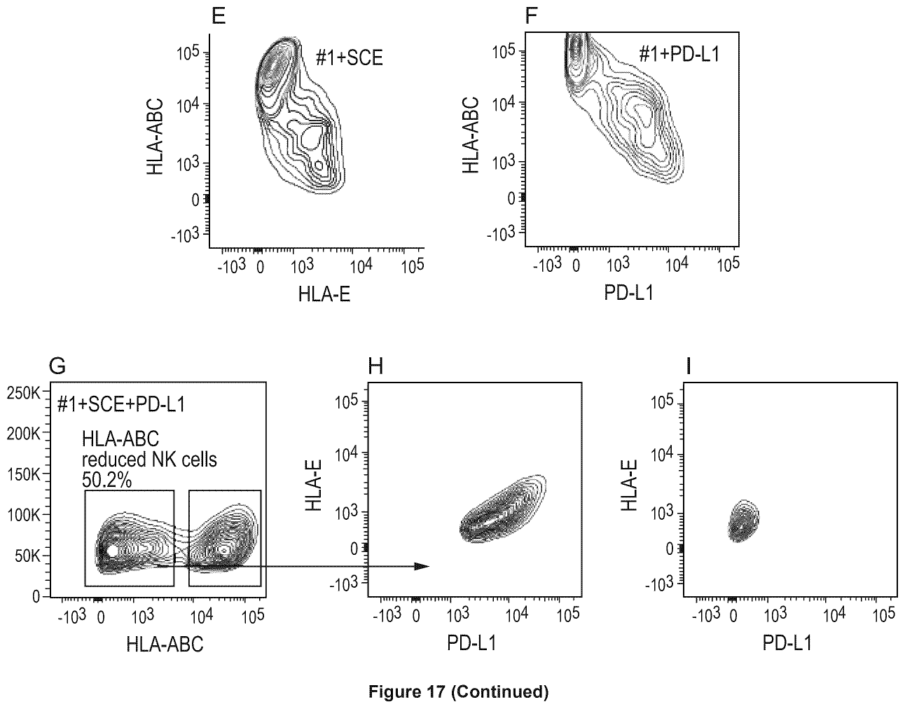

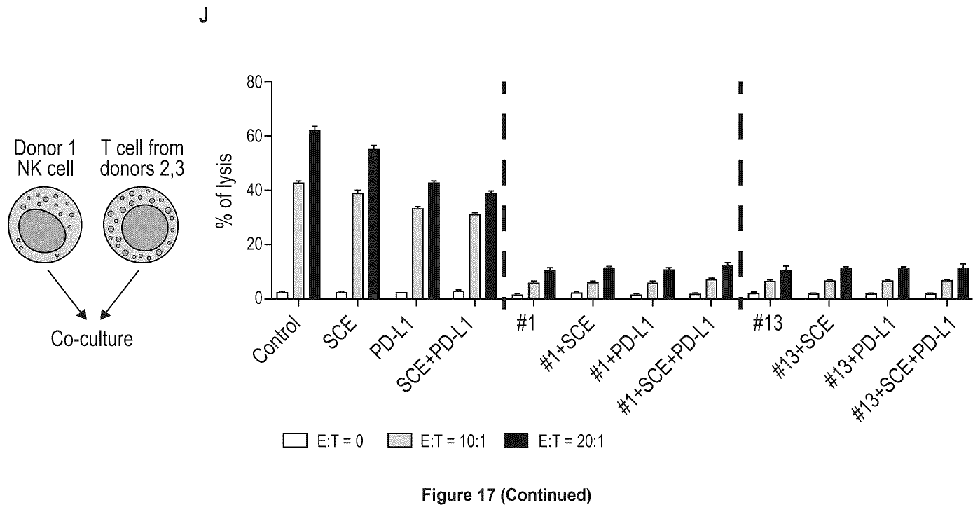

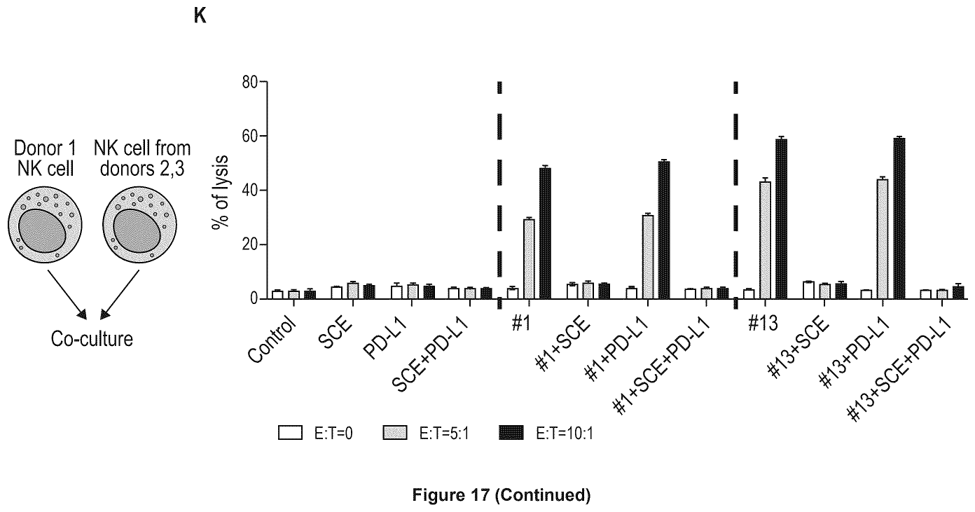

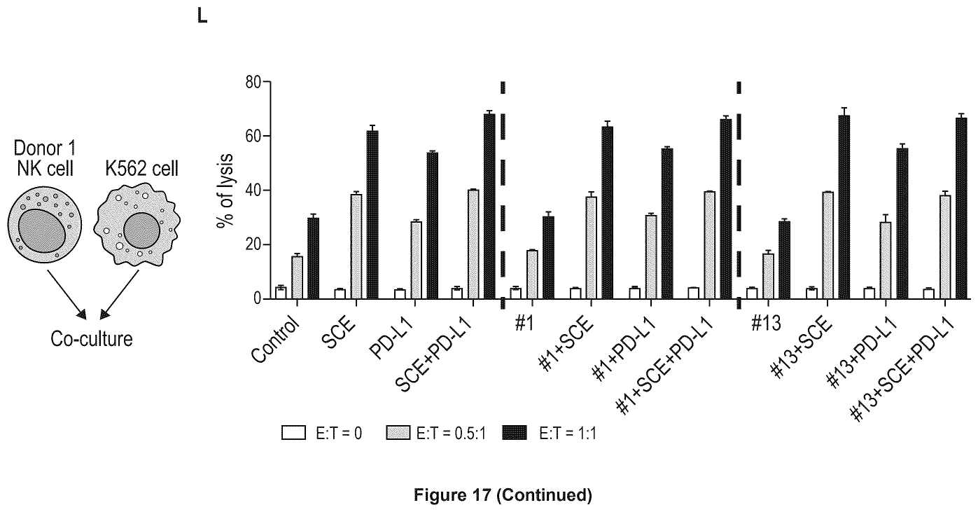

The combination of HLA-ABC knockdown and exogenous PD-L1 and/or HLA-E expression to inhibit killing of allogeneic NK cells by host T cells and NK cells was tested (FIG. 15). Donor 1 NK cells were transduced with lentivectors expressing shRNA #1, shRNA #13, SCE, PD-L1 or various combinations and downregulation of HLA-ABC, and/or expression of HLA-E or PD-L1 were verified (FIG. 17a-b). For example, transduction of NK cells with a lentivector expressing shRNA #1 plus both SCE and PD-L1 yielded a population of transduced NK cells with reduced HLA-ABC expression but expressing both SCE and PD-L1 (FIG. 17c-d). Transduction of NK cells with lentivectors expressing both shRNA #1 plus PD-L1 or SCE yielded a population of transduced NK cells that were reduced in HLA-ABC expression but overexpressed SCE or PD-L1, respectively (FIG. 17e-f). Similarly, transduction of NK cells with a lentivector expressing shRNA #1 plus both SCE and PD-L1 yielded a population of transduced NK cells that were reduced in HLA-ABC expression but overexpressed both SCE and PD-L1 (FIG. 17G-I).

The transduced NK cells were sorted, expanded and used as targets. CD8+ T cells from donors 2 and 3 (HLA-A*02:01−) were primed with mitomycin C pretreated non-transduced NK cells from donor 1 (HLA-A*02:01+) in the presence of IL-2 for 7 days. Primed CD8+ T cells were then cocultured with labeled untransduced (control) or transduced NK cells from donor 1 at E:T ratio of 0, 10 and 20 overnight (FIG. 17j). Untransduced NK cells from donor 1 were efficiently lysed by CD8+ T cells from donors 2 and 3, reaching 60% lysis at E:T ratio of 20 (FIG. 17j). Knockdown of HLA-ABC by shRNA #1 or #13 reduced NK cell killing to ˜10% at E:T ratio of 20. Expression of PD-L1 or SCE only moderately inhibited NK cell killing by CD8+ T cells, with PD-L1 being more effective than SCE (40% vs 55%, FIG. 17j). Combination of knockdown of HLA ABC by shRNA #1 or #13 plus SCE and/or PD-L1 expression reduced NK cell killing to 10% at E:T ratio of 20, approximately the same levels by HLA-ABC knockdown alone. Thus, HLA-ABC knockdown in allogeneic NK cells is the most critical for escaping host CD8+ T cell killing in vitro, whereas overexpression of PD-L1 and/or SCE is less effective on its own.

To test whether HLA-ABC knockdown plus SCE and/or PD-L1 expression in allogeneic NK cells confers resistance to NK cell killing, NK cells from donors 2 and 3 were cocultured with labeled untransduced (control) or transduced NK cells from donor 1 at E:T ratio of 0, 5 and 10 for 6 hours in the presence of anti-CD47 and Fc blocker (FIG. 17k). Untransduced NK cells or HLA-E and/or PD-L1 expressing donor 1 NK cells were resistant to lysis by NK cells from donors 2 and 3 (FIG. 17k), whereas knockdown of HLA-ABC by shRNA #1 or #13 led to effective NK cell killing, reaching 50% and 60%, respectively, at E:T ratio of 10. Expression of SCE (or plus PD-L1) completely abolished killing of HLA-ABC-reduced allogeneic NK cells, whereas expression of PD-L1 did not inhibit NK cell killing. Thus, exogenous expression of SCE, but not PD-L1, potently inhibits the killing of HLA-ABC-reduced allogeneic NK cells by host NK cells.

The cytotoxicity of NK cells that had reduced HLA-ABC expression but overexpressed SCE and/or PD-L1 was also evaluated (FIG. 17l). Untransduced and transduced NK cells from donor 1 were cocultured with K562 cells at E:T ratios of 0, 0.5 and 1 for 4 hours. Untransduced NK cells killed K562 cells in a dose-dependent manner (FIG. 17l). Knockdown of HLA-ABC did not significantly affect NK cell killing of K562 cells. Unexpectedly, expression of SCE or PD-L1 with or without HLA-ABC knockdown significantly increased killing of K562 cells (compared 30% vs 60-70% lysis at E:T ratio of 1). In particular, expression of SCE enhanced NK cell cytotoxicity significantly more than expression of PD-L1.

Together, these results show that in vitro i) HLA-ABC knock down in allogeneic NK cells is more effective than overexpression of SCE and/or PD-L1 in inhibiting killing by host CD8+ T cells, ii) overexpression of SCE in allogeneic NK cells is more effective than overexpression of PD-L1 in inhibiting host NK cell killing due to loss of HLA-ABC, and iii) overexpression of SCE and PD-L1 significantly enhances NK cell killing of K562 target cells.

### Combination of HLA-ABC Knockdown and PD-L1 or HLA-E Expression in Allogeneic NK Cells Confers Resistance to Host Immune Cell Rejection In Vivo

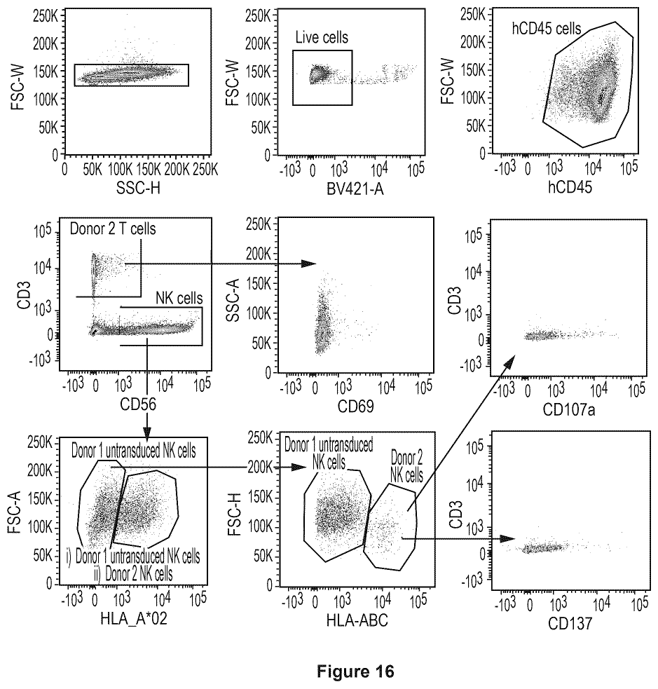

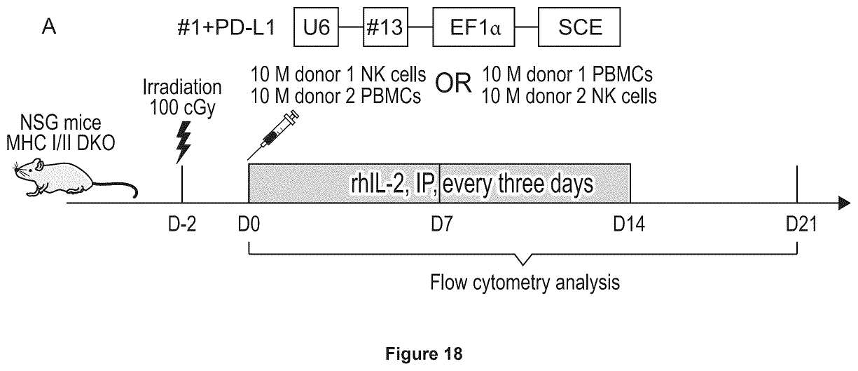

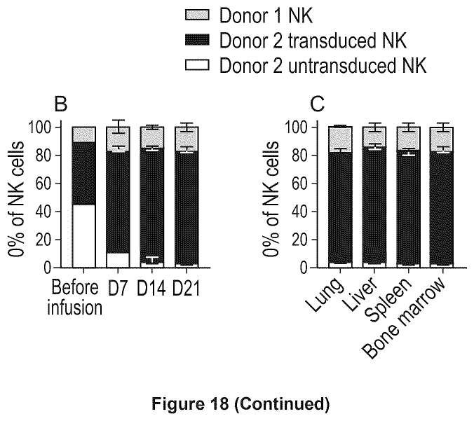

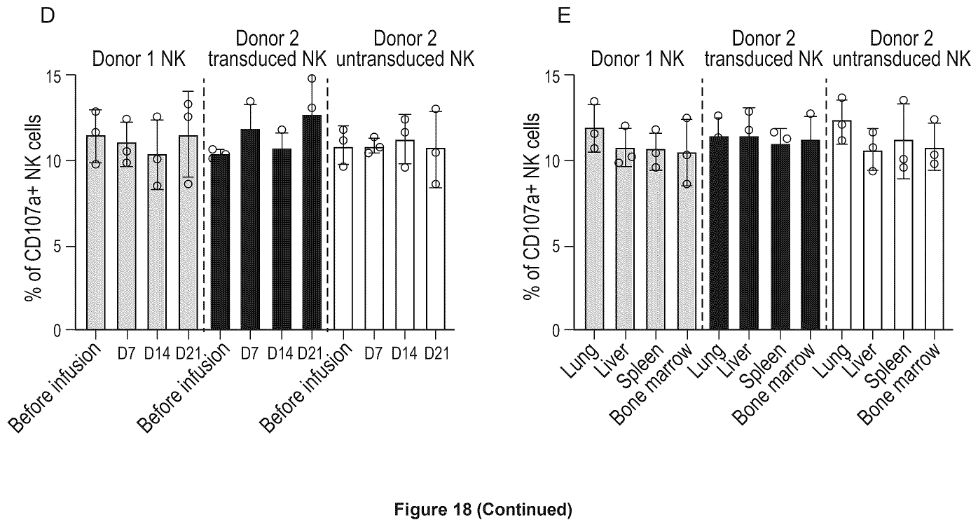

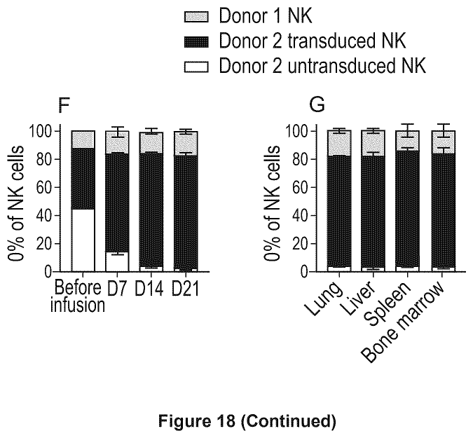

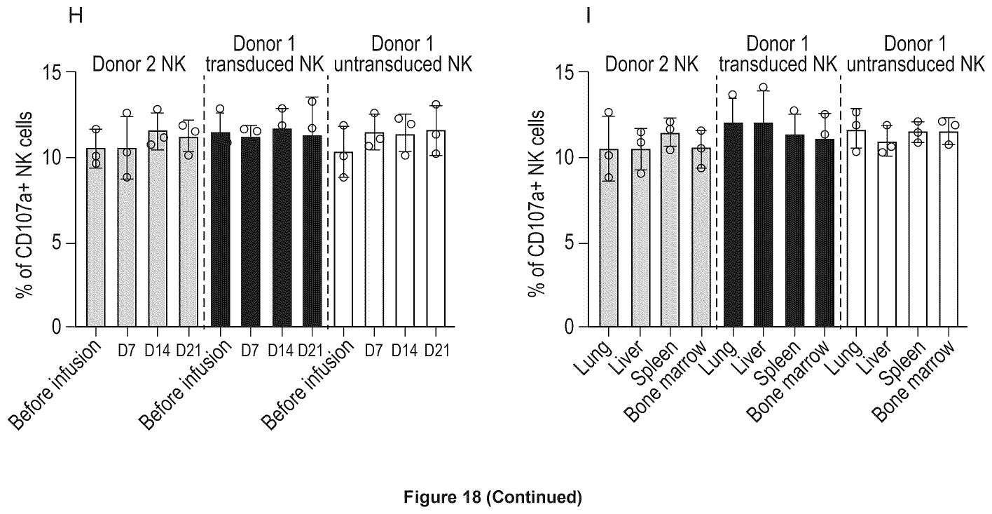

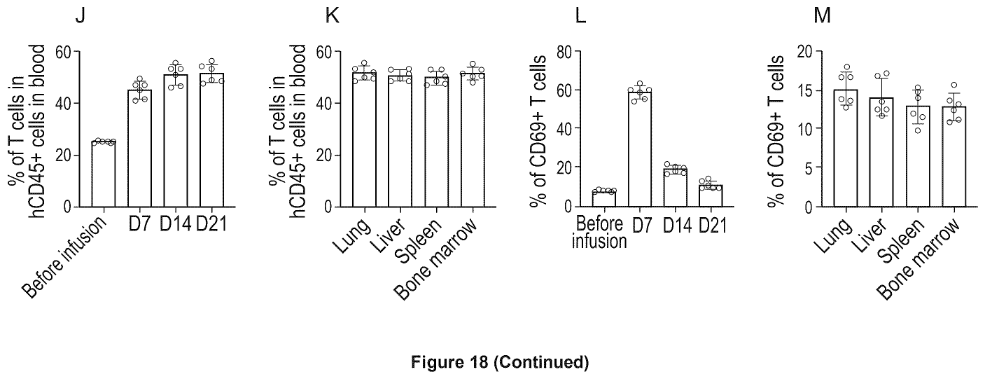

Whether allogeneic NK cells with HLA-ABC knockdown but PD-L1 or SCE overexpression are resistant to host T cell and NK cell rejection in vivo was tested. In the first set of experiments, purified NK cells from donor 1 (HLA-A*02:01+) were transduced with the lentivector expressing shRNA #13 and SCE (FIG. 18a). The transduced and untransduced NK cells were mixed with equal numbers of PBMCs from donor 2 (HLA-A*02:01−) and adoptively transferred into NSG mice that were also deficient in both MHC classes I and II and had been irradiated 2 days earlier. Human NK cells and T cells in the cell mixture before adoptive transfer, in peripheral blood 7, 14 and 21 days after transfer, and in lung, liver, spleen and bone marrow 21 days after transfer were analyzed by flow cytometry (FIG. 18a). Transduced NK cells from donor 2 were identified as CD45+, CD56+, HLA-ABC-reduced, HLA-E+, and HLA-A*02:01−; untransduced NK cells from donor 2 were identified as CD45+, CD56+, HLA-ABC+, HLA-E−, and HLA-A*02:01+, and NK cells from donor 1 PBMC were identified as CD45+, CD56+, HLA-ABC+, HLA-E−, and HLA-A*02:01 (FIG. 16). Before adoptive transfer, the relative percentages of transduced and untransduced NK cells from donor 2 among total NK cells were ˜45% each, whereas the percentages of donor 1 NK cells were ˜10% (FIG. 18b). The percentages of donor 2 transduced NK cells increased to ˜70% 7 days post transfer and to ˜80% 14- and 21-days post transfer in the blood. Correspondingly, the percentage of donor 2 untransduced NK cells decreased to ˜10% 7 days post transfer and to <5% 14- and 21-days post transfer, whereas the percentages of donor 1 NK cells increased to ˜20%. Consistently, the percentages of donor 2 transduced NK cells were ˜80% in lung, liver, spleen and bone marrow at 21 days post transfer, whereas the percentages of donor 2 untransduced NK cells were <2% in these tissues (FIG. 18c). There was no significant change in CD107a expression by donor 2 NK cells and donor 1 NK cells (transduced or untransduced) before adoptive transfer and 7, 14 and 21 days after adoptive transfer in the peripheral blood or tissues (FIG. 18d-e). Thus, the untransduced NK cells (HLA-ABC+SCE−), but not transduced NK cells (HLA-ABC−/low SCE+), from donor 2 are eliminated.

The relative levels of T cells and their activation status as indicated by expression of CD69 in the recipient mice were assayed. The percentages of T cells within human CD45+ cells were ˜25% in the cell mixture before adoptive transfer and few expressed CD69 (FIG. 18j-m). The percentages of T cells increased to 45-50% among human CD45+445 cells in the peripheral blood on day 7, 14 and 21 post transfer and to ˜50% in the tissues (FIG. 18k). Notably, the percentages of CD69+ T cells increased to ˜60% 7 days post transfer and then decreased to ˜20% and ˜10% 14- and 21-days post transfer in the blood, respectively, and to ˜12% in the tissues (FIG. 18m).

A reciprocal experiment was performed where NK cells from donor 1 were transduced, mixed with PBMCs from donor 2, and adoptively transferred into the same recipient mice. Similar results were observed, i.e., preferential elimination of donor 2 untransduced NK cells (HLA-ABC+PD-L1) from donor 1 and expansion of transduced NK cells (HLA-ABC-reduced PD-L1) from donor 1 in the recipient mice (FIG. 18f-i).

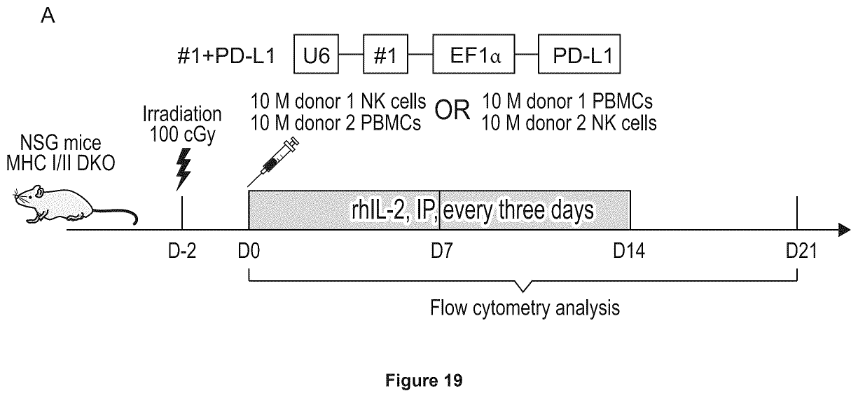

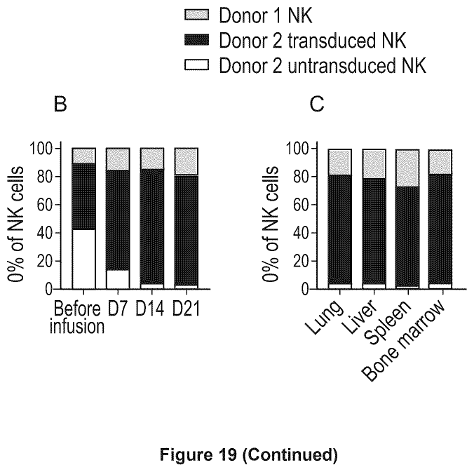

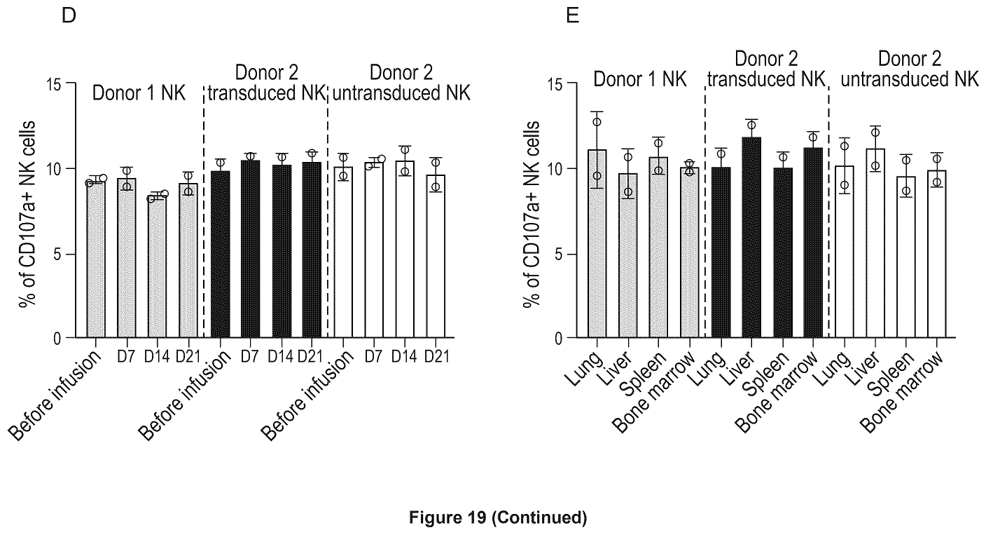

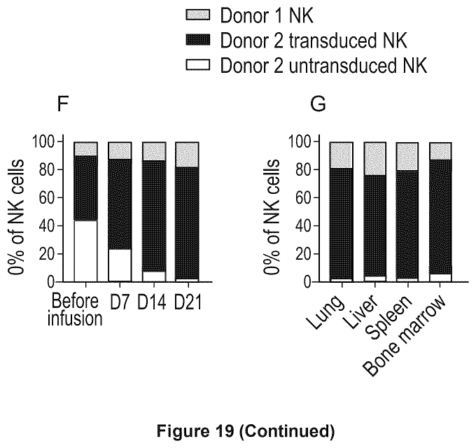

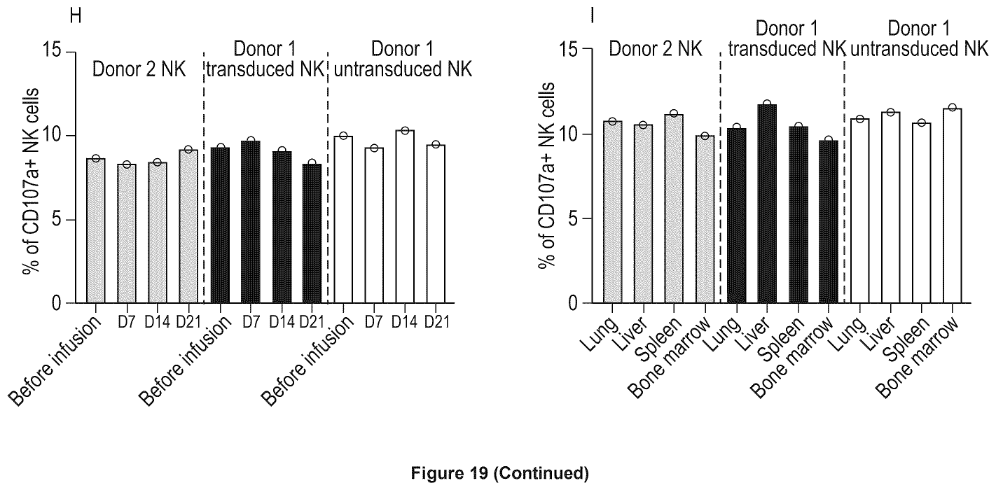

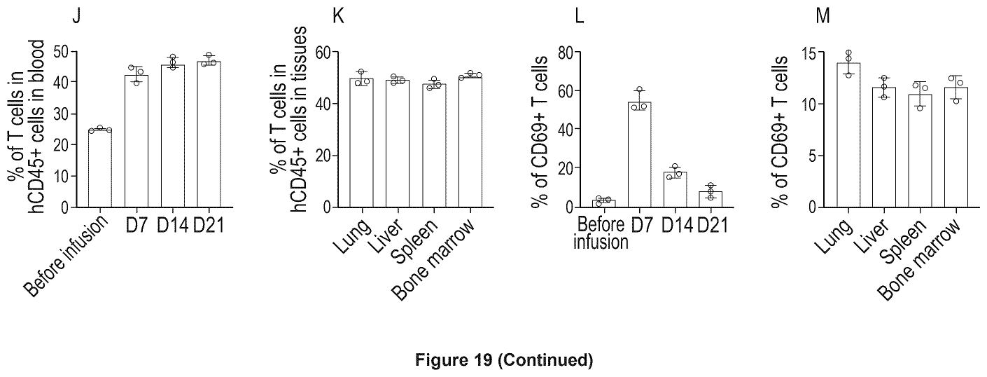

In a similar reciprocal experiment, NK cells from donor 2 were transduced with lentivector expressing shRNA #1 plus PD-L1. NK cells, containing both transduced and untransduced cells, from donor 2 were mixed with an equal number of PBMCs from donor 1 and adoptively transferred into NSG mice lacking MHC classes I and II. Again, similar results were obtained, i.e., i) allogeneic untransduced NK cells (HLA-ABC+PD-L1− were rejected, whereas allogeneic transduced NK cells (HLA-ABC-reduced PD-L1+) were not, and ii) induction of highest percentages of CD69 expression by T cells 7 days post transfer (FIG. 19). Together, these results show that simultaneous knockdown of HLA ABC and exogenous expression of either SCE or PD-L1 in allogeneic NK cells confer resistance to rejection by host T cells and NK cells in vivo.

CAR NK Cells with HLA-ABC Knockdown and PD-L1 or SCE Expression not Only Resist Rejection but Also Exhibit Enhanced Cytotoxicity Against Tumor Cells In Vitro

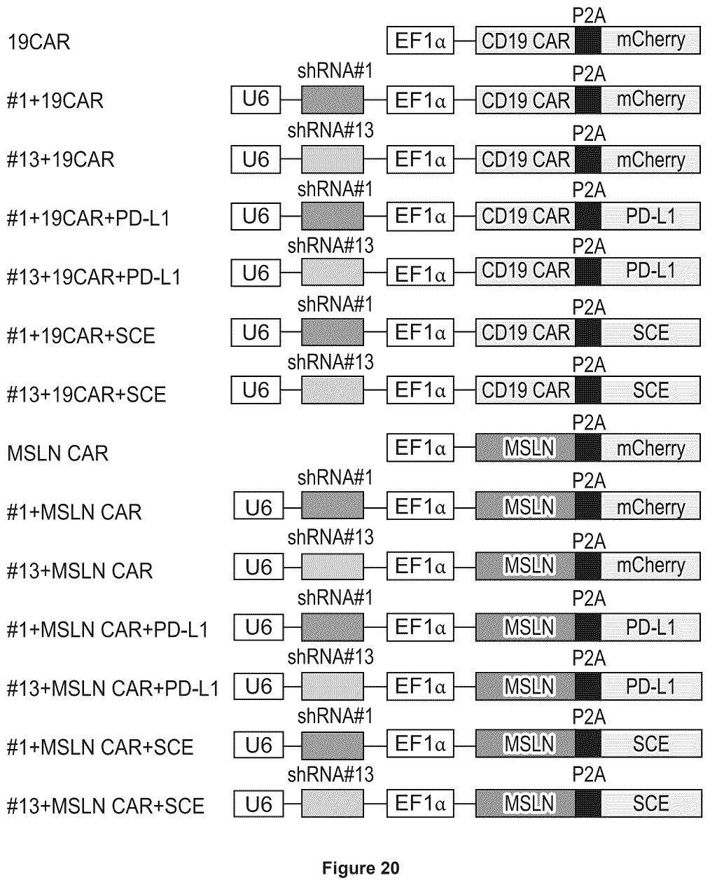

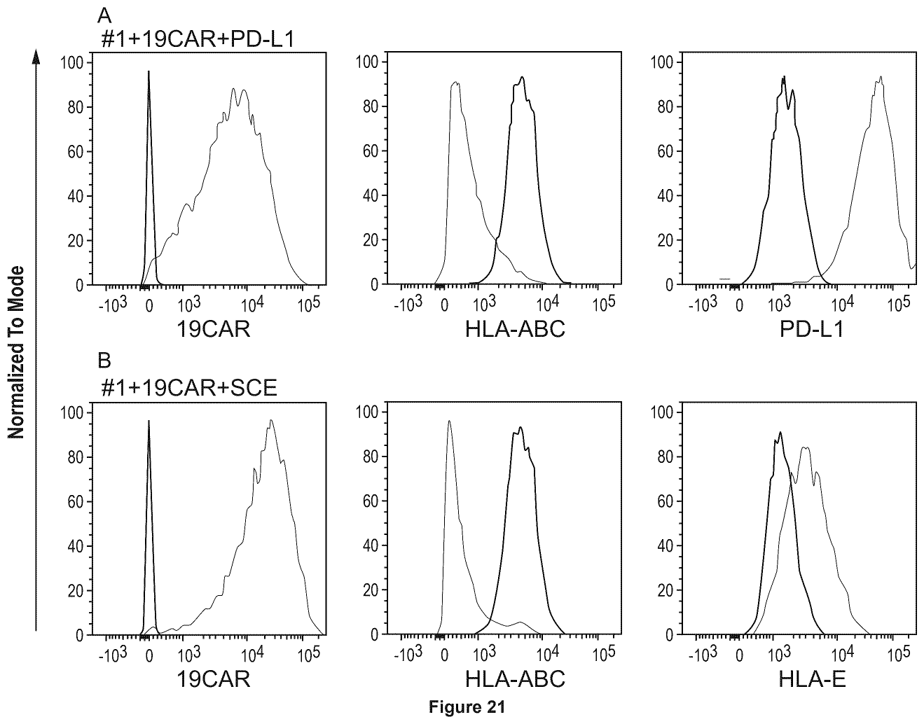

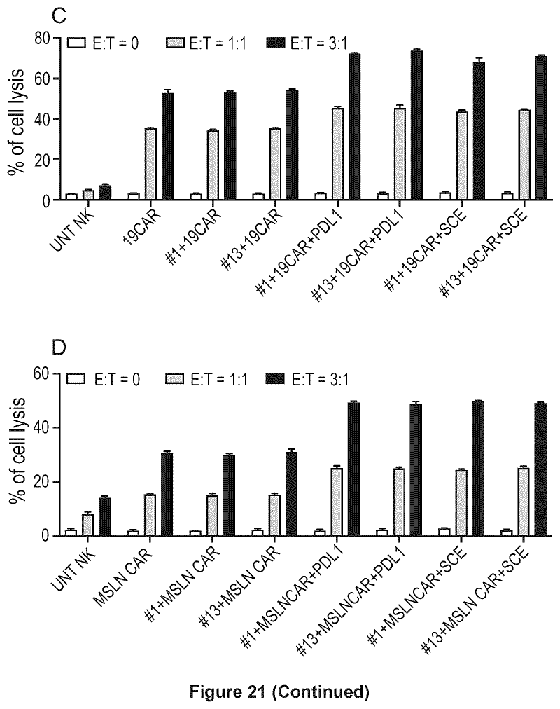

To investigate whether HLA-ABC knockdown and exogenous expression of PD-L1 or SCE could be used in one-step production of allogeneic CAR-NK cells, lentivectors encoding shRNA #1 or #13 were constructed, CD19-CAR or mesothelin (MSLN)-CAR, and PD-L1 or SCE (FIG. 20). NK cells were transduced with these lentivectors and expression of CAR, SCE or PDL1, and HLA-ABC were verified. For example, NK cells that were transduced with lentivector expressing shRNA #1 (referred to as #1), MSLN CAR, and PD-L1 or SCE expressed MSLN CAR and PD-L1 or SCE and had reduced HLA-ABC expression (FIG. 21a-b), suggesting that shRNA, CAR and PD-L1 or SCE can be expressed from the same lentivector.

To test the CAR-mediated target cell killing, the killing of OVCAR8 cells by MSLN-CAR NK cells and Raji cells by CD19-CAR NK cells in vitro was assayed. MSLN-CAR NK cells killed OVCAR8 cells in a dose-dependent manner, reaching 30% at E:T ratio of 3 (FIG. 21d). Knocking down of HLA ABC by shRNA #1 or #13 in MSLN-CAR NK cells did not have any significant effect on CAR mediated killing of OVCAR8 cells. Expression of PD-L1 or SCE in MSLN-CAR-NK cells with HLA ABC knockdown killed OVCAR8 cells more effectively, reaching ˜50% lysis at E:T ratio of 3.

Similarly, untransduced NK cells did not kill Raji cells appreciably, whereas CD19-CAR NK cells efficiently killed Raji cells in a dose-dependent manner, reaching ˜50% at E:T ratio of 3 (FIG. 21c). Knocking down of HLA-ABC by shRNA #1 or #13 in CD19-CAR NK cells did not have any significant effect on CAR-mediated killing of Raji cells. In contrast, CD19-CAR NK cells that had HLA-ABC knockdown and exogenously expressed PD-L1 or SCE killed Raji cells more effectively, reaching ˜80% lysis at E:T ratio of 3, consistent with enhanced killing of K562 cells by NK cells that express PD-L1 or SCE (FIG. 21d). These results show that shRNA knockdown of HLA-ABC and exogenous expression of PD-L1 or SCE can be integrated into the existing CAR lentivector in a one-step production of allogeneic CAR NK cells, and exogenous PDL1 or SCE expression enhances CAR-NK cell killing of target cells.

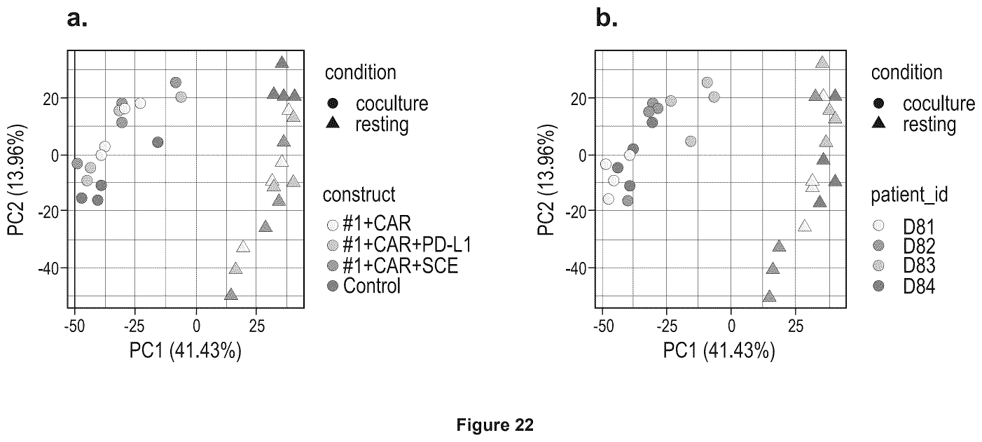

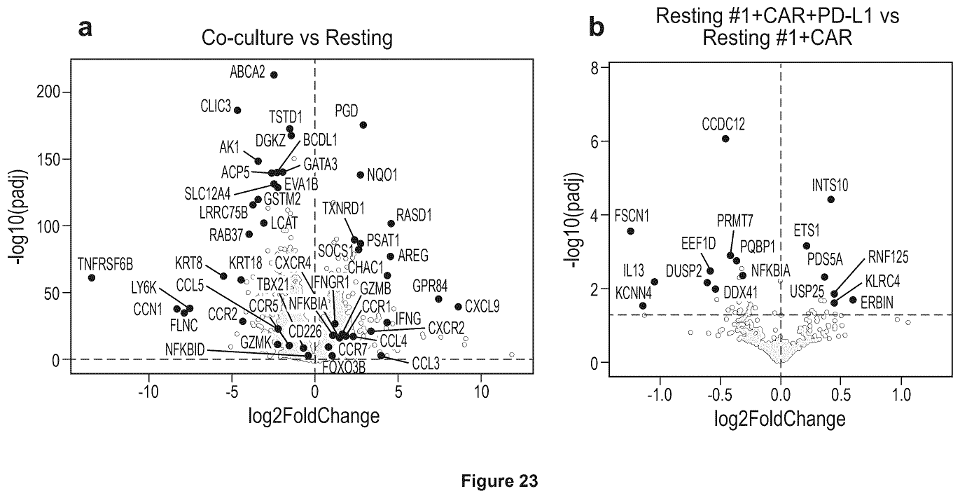

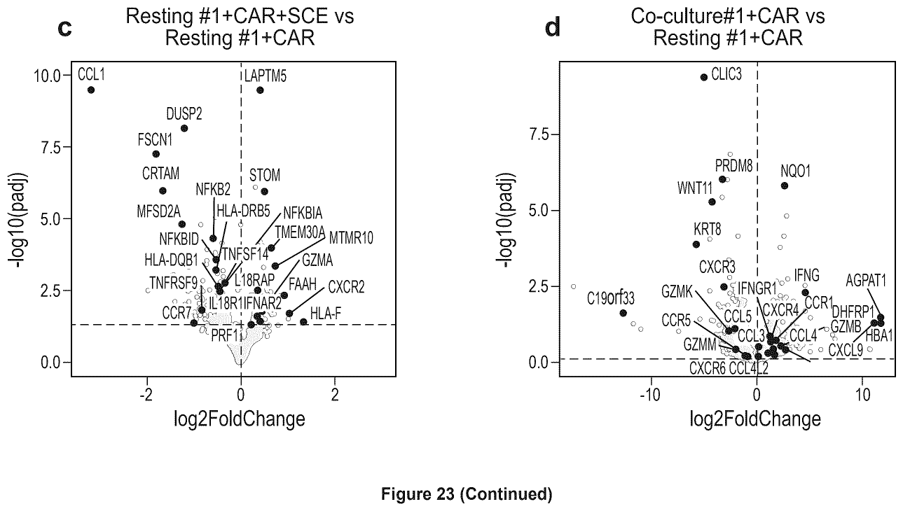

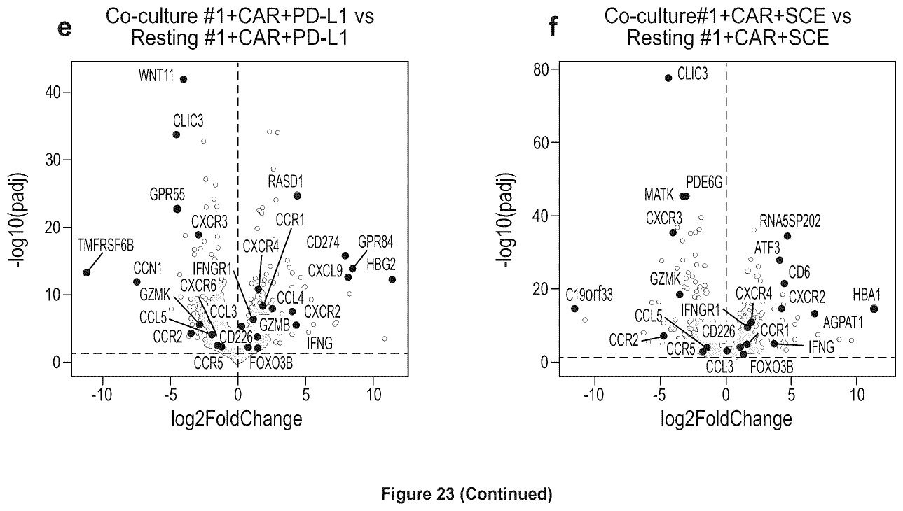

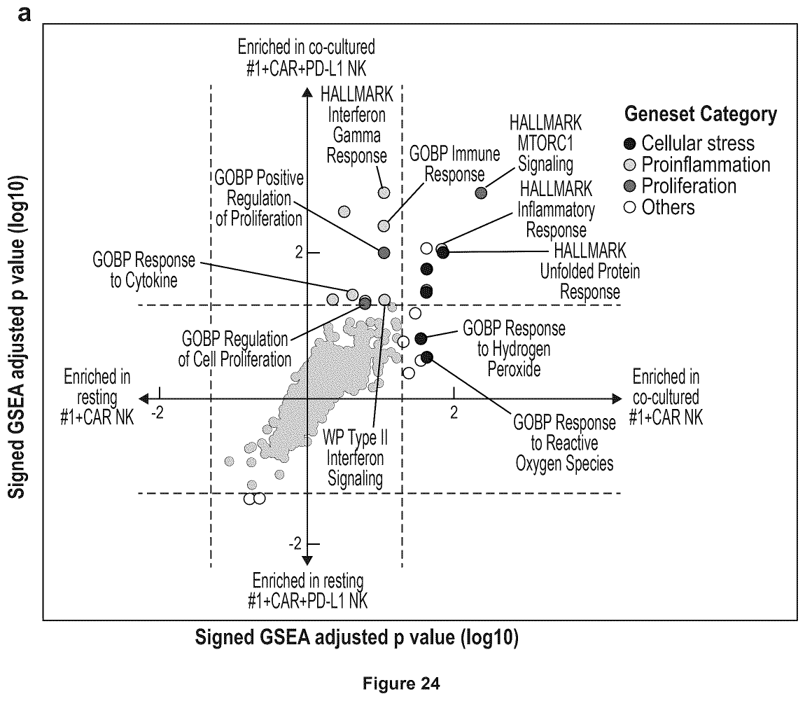

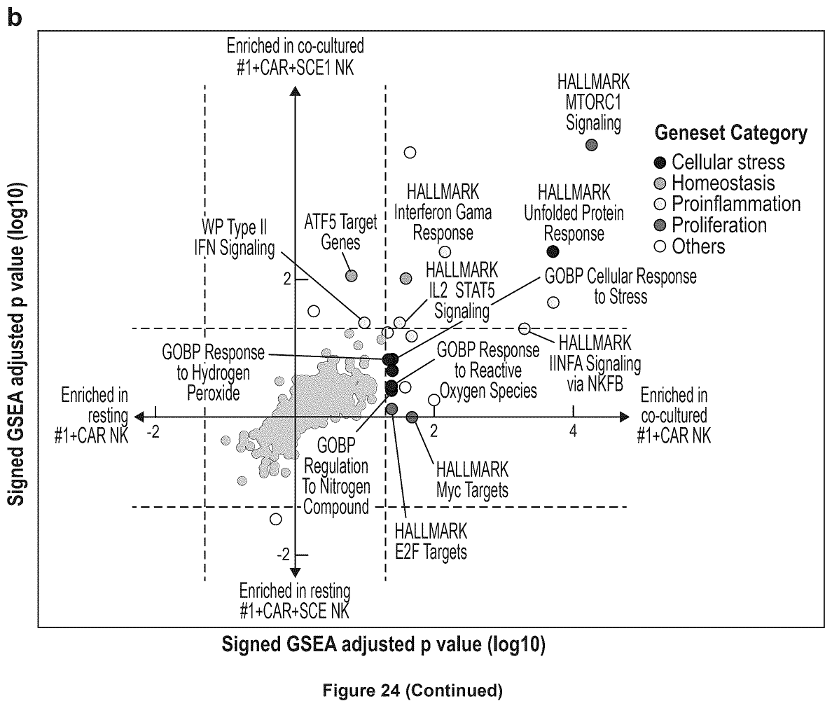

To elucidate the mechanisms underlying the enhanced NK cell cytotoxicity following PD-L1 and SCE overexpression, a comprehensive RNAseq analysis was done. NK cells isolated from 4 unrelated donors were transduced with lentivectors expressing shRNA #1+MLSN CAR, shRNA #1+MLSN CAR+PD-L1, or shRNA #1+MLSN CAR+SCE. NK cells from the same donors but without transductions were used as controls. NK cells were labeled with CTV and then cocultured with OVCAR8 target cells or in the media alone for 18 hours in triplicates. The cocultured NK cells were purified by cell sorting and processed for RNAseq. The principal component analysis (PCA) revealed that NK cells coculturing with OVCAR8 target cells account for the greatest transcriptional difference among the samples (FIG. 22a-b). The differential gene expression analysis revealed that the cytotoxicity-related genes (e.g., GZMB and IFNG) were upregulated in NK cells after coculture with target cells (FIG. 23a). The noncocultured (resting) #1+CAR+PD-L1 NK cells upregulated KLRC4 but downregulated anti-inflammatory cytokine IL-13 compared with resting CAR NK cells. Similarly, the #1+CAR+SCE NK cells expressed higher levels of PRF1, GZMA, and IFNAR2 compared with the resting #1+CAR NK cells (FIG. 23b-c). Furthermore, cocultured #1+CAR, #1+CAR+PDL1, and #1+CAR+SCE NK cells up-regulated cytotoxicity-related genes (e.g., CD226, IFNG, IFNGR1, and GZMB) compared to their resting counterparts (FIG. 23d-f), suggesting that CAR recognition of target cells is a superior signal to activate NK cells. The pathway enrichment analysis revealed that the proliferation and proinflammation pathways were enriched in #1+CAR+PD-L1 NK cells, and homeostasis pathways were enriched in #1+CAR+SCE NK cells after co-culture with the target cells. In contrast, the cellular stress pathways were more enriched and associated with #1+CAR NK cells after co-culture with the target cells (FIG. 24a-b). This data suggests that CAR NK cells may experience more cellular stress when combating target tumor cells, which is alleviated by exogenous expression of inhibitory ligands PD-L1 or SCE.

Allogeneic CD19-CAR NK Cells with HLA-ABC Knockdown and PD-L1 or SCE Overexpression are Resistant to Host Immune Cell Killing and Control Tumor Growth In Vivo

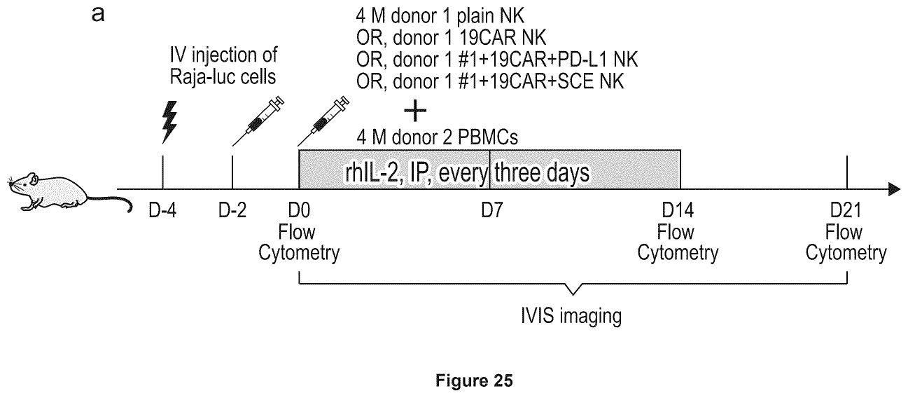

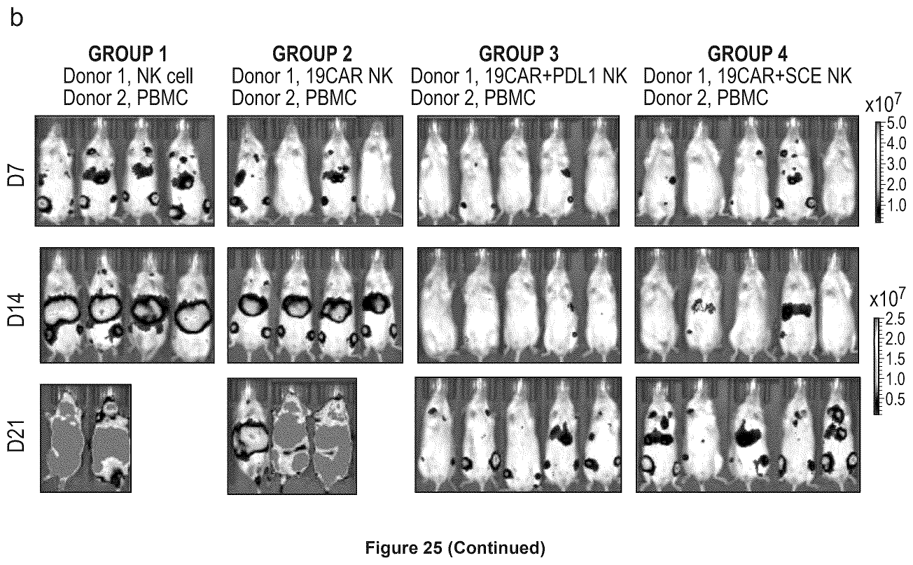

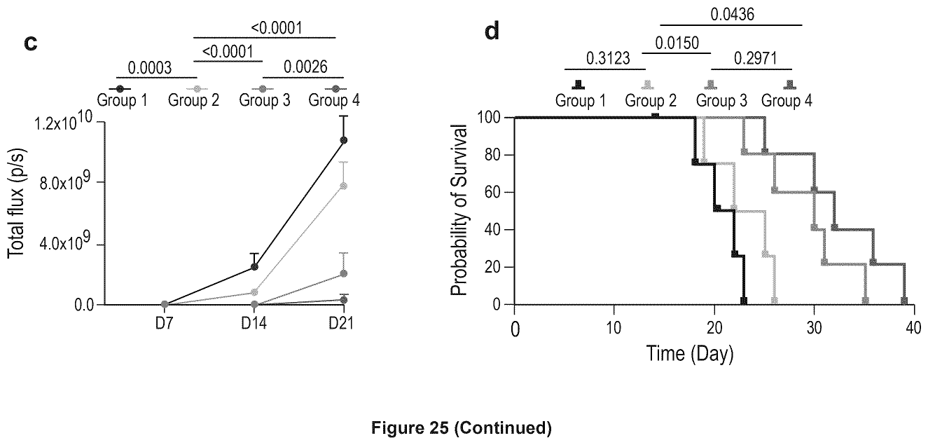

To determine whether CAR NK cells with HLA-ABC knockdown and PD-L1 or SCE overexpression could control tumor growth and avoid rejection by host T and NK cells in NSG mice, purified NK cells from donor 1 (HLA-A2*02:01+) were either not transduced (group 1) or transduced with lentivectors expressing CD19 CAR (group 2), #1+CD19 CAR+PD-L1, (group 3) and #1+CD19 CAR+SCE (group 4) (FIG. 25a-b). The donor 1 NK cells were mixed with equal numbers of PBMCs from donor 2 (HLA-A*02:01−) and adoptively transferred into NSG mice that had been injected with Raji-luciferase cells two days earlier. Tumor burden was significantly reduced in groups 3 and 4 mice as compared to groups 1 and 2 mice as indicated by significantly lower bioluminescence (BLI) and longer survival (FIG. 25b-d). Notably, group 3 mice showed a significantly lower BLI and slightly better overall survival than group 4 mice (FIG. 25c-d).

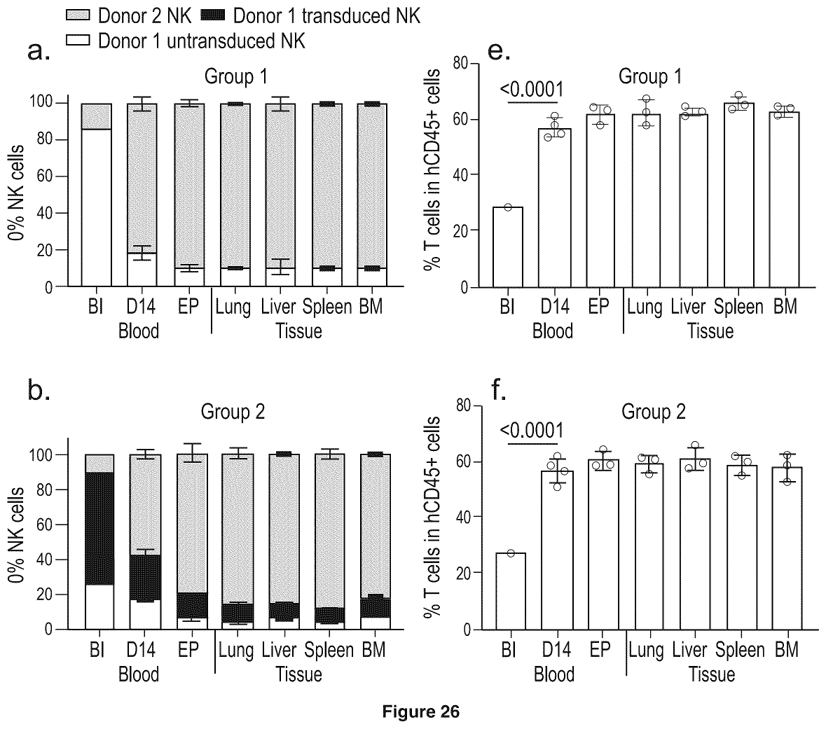

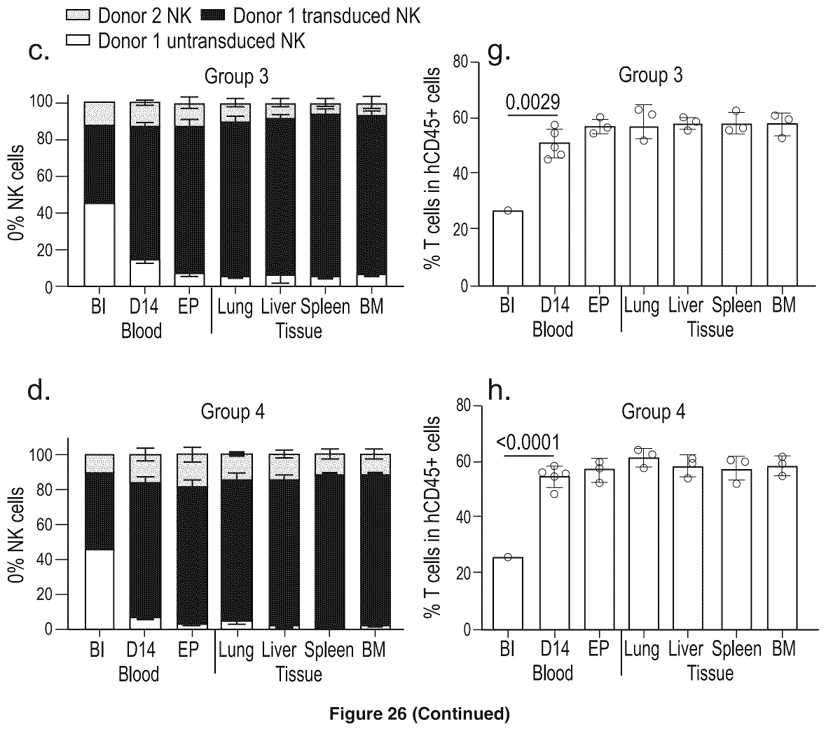

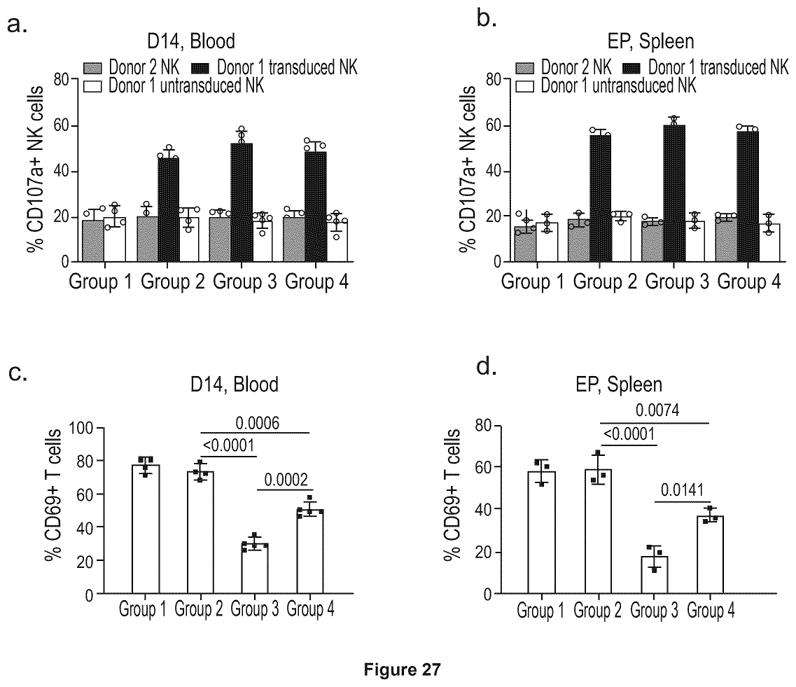

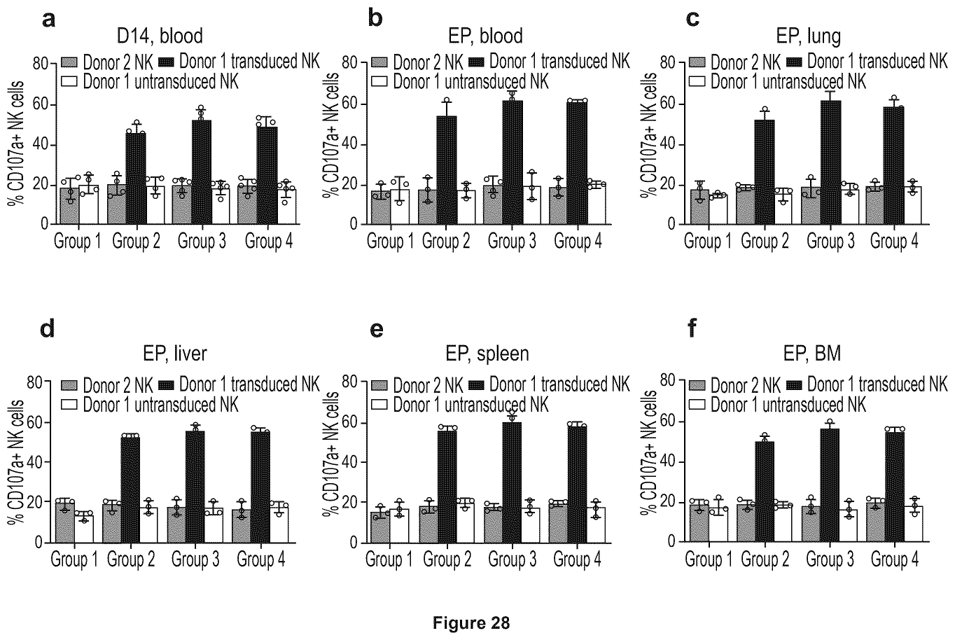

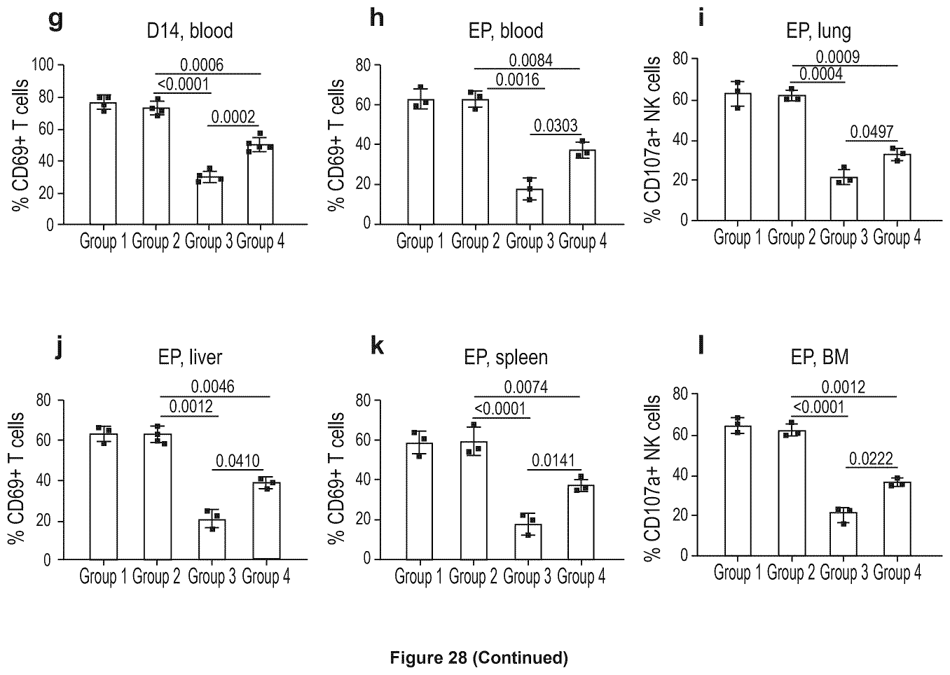

It was further analyzed that the proportion and activation status of NK and T cells in the tumor-bearing recipient mice. In group 1 mice, untransduced donor 1 NK cells were mixed with donor 2 PBMCs and adoptively transferred into tumor bearing recipient mice. As expected donor 1 NK cells were rejected by donor 2 T cells as indicated by i) rapid disappearance of donor 1 NK cells from 87.8% of total NK cells before adoptive transfer to ˜20% in the blood 14 days after transfer and to ˜10% in the tissues at the endpoint (FIG. 26a and FIG. 28a-f), ii) increase of donor 1 T cells from −30% before adoptive transfer to ˜60% in the blood 14 days after transfer and in the tissues at the endpoint (FIG. 26e), and iii) ˜75% of T cells were CD69+ in the blood at 14 days after transfer and ˜60% in the tissues at the endpoint (FIG. 27c-d and FIG. 28g-1).

In group 2 mice, untransduced and CD19 CAR transduced donor 1 NK cells were mixed with donor 2 PBMCs and adoptively transferred into tumor bearing recipient mice. Again, the donor 1 NK cells were rejected by donor 2 T cells as indicated by i) rapid disappearance of both transduced and untransduced donor 1 NK cells from 40%-47% before adoptive transfer to 18%-25% in the blood 14 days after transfer and to ˜10% in the tissues at the endpoint (FIG. 26b and FIG. 28a-f), ii) increase of donor 1 T cells from ˜30% before adoptive transfer to ˜60% in the blood 14 days after transfer and in the tissues at the end point (FIG. 26f), and iii) ˜75% of T cells were CD69+ in the blood at 14 days after transfer and ˜60% in the tissues at the end point (FIG. 27c-d, 26h and FIG. 28g-1). Notably, significantly higher percentages of donor 1 transduced CAR NK cells (˜50%) were CD107a+ than donor 1 untransduced NK cells (˜20%) (FIG. 27a-b and FIG. 28a-f), suggesting their response to Raji target cells.

In groups 3 and 4 mice, the relative percentages of donor 1 transduced and untransduced NK cells were ˜45% before adoptive transfer (FIG. 26c-d). The percentages of transduced NK cells increased to ˜75% in the blood 14 days after transfer and to ˜90% in the tissues at the end point, whereas the percentages of untransduced NK cells decreased to ˜10% in the blood 14 days after transfer and to <5% in the tissues at the end point. The relative percentage of donor 2 NK cells was ˜10% before adoptive transfer, and this percentage did not change significantly in the blood or tissues at the end point. Significantly higher percentages of donor 1 transduced CAR NK cells (50-60%) were CD107a+ than donor 1 untransduced NK cells (˜20%) (FIG. 27a-b and FIG. 28a-f). The relative percentage of donor 2 T cells within CD45+ human cells was ˜25% before adoptive transfer, and this percentage increased to ˜60% in the blood 14 days after transfer and in the tissues at the end point (FIG. 26g-h). In contrast, only ˜20% (group 3) and ˜40% (group 4) T cells were CD69+ in the blood at 14 days after transfer and in the tissues at the end point (FIG. 26g-h and FIG. 28g-1), suggesting inhibition of donor 2 T cells by allogeneic donor 1 CAR NK cells with HLA-ABC knockdown and PD-L1 or SCE expression. These results show that i) allogeneic CAR NK cells with HLA-ABC knockdown and HLA-E or PDL1 expression can be produced in one-step lentiviral transduction; ii) allogeneic CAR NK cells with HLA-ABC knockdown and SCE and/or PD-L1 expression are resistant to rejection by host T and NK cells in vivo; and iii) allogeneic CAR NK cells with HLA-ABC knockdown and SCE and/or PD-L1 expression exhibit enhanced tumor control in a CAR-dependent manner.

Thus, described herein is a specific shRNA that is effective in knocking down surface expression of the most prevalent HLA-ABC alleles, including HLA-A*02:01, without affecting HLA-E expression due to two nucleotide mismatches between shRNA and HLA E heavy chain (FIG. 11a and Table 2). Because shRNA-mediated knockdown is less complete as compared to CRISPR-mediated knockout, HLA-E and/or PD-L1 were simultaneously expressed to enhance allogeneic NK cells resistance to rejection by host T cells and NK cells. To simplify HLA-E expression and identify the most potent inhibitory peptide, three single-chain HLA-E (SCE) variants were tested and different peptides derived from the leader sequences of HLA-ABC and HLA-G (FIG. 14a-f). The results show that SCE Y84C, which is expressed at the highest level, presenting the peptide from the HLA-G leader sequence is the most potent in inhibiting NK cell reactivity.

By combining the novel shRNA, CD19 or MSLN CAR, PD-L1 or SCE into the same lentivector, production of allogeneic CAR-NK cells from peripheral blood NK cells by one-step lentiviral transduction is possible and show that these allogenic CAR-NK cells are resistant to rejection by host immune system (T cells, NK cells and macrophages) and can kill target tumor cells in a CAR dependent manner in vivo. While allogeneic NK cells were rapidly rejected by host T cells in NSG recipient mice as indicated by simultaneous disappearance of allogeneic NK cells and activation (CD69) and expansion of T cells at 7 days post transfer (FIGS. 18a,b-g, and j-m and FIG. 25-27), allogeneic (CAR−) NK cells with HLA-ABC knockdown and expression of PD-L1 or SCE were not rejected. In tumor bearing mice, significantly higher proportions of allogeneic CAR-NK were activated as indicated by CD107a expression, leading to better control of tumor burden and survival (FIG. 25-27). Correlating with more potent inhibition of T cell rejection in vitro, expression of PD-L1 in combination with HLA-ABC knockdown and CAR expression is more effective than expression of SCE for tumor control and survival in vivo, suggesting HLA-ABC knockdown and PD-L1 expression is the most potent combination for inhibiting T cell rejection of allogenic human NK cells. Overexpression of PD-L1 has also been used to avoid the rejection of allografts, particularly the transplanted islets, to treat type I diabetes. Overexpression of PD-L1 on allogeneic NK cells for inhibiting T cell rejection was shown, including NK cells, without stimulation, are PD-L1 negative or low. PD-L1 overexpressed allogeneic NK cells can moderately inhibit host T cell responses. These findings suggest that PD-L1 alone on immune and likely non-immune cells is insufficient to inhibit allogeneic immune responses completely, and other engineering, such as HLA-ABC knockdown, should be combined to achieve better survival of engineered cells in allogeneic hosts. Unexpectedly, the exogenous expression of PD-L1 or SCE significantly enhances NK cell killing of K562 cells in the absence of CAR (FIG. 17l) and Raji and OVCAR8 tumor cells in a CAR-dependent manner (FIGS. 21 and 24). Comparison of transcriptional profiles of unmodified NK cells (no CAR) and CAR NK cells with or without coculture with target OVCAR8 cells revealed the upregulation of genes involved in NK cell cytotoxicity following PD-L1 or SCE expression. In particular, following coculture with OVCAR8 target cells, MSLN CAR NK cells upregulated genes involved in stress responses, which was inhibited by exogenous PD-L1 or SCE expression. Thus, described herein is a one-step approach to construct allogeneic CAR NK cells with reduced HLA-ABC expression but PD-L1 or SCE overexpression. The resulting allogeneic CAR NK cells are resistant to host immune cell rejection while mediating enhanced anti-tumor responses and thus represent a significant advancement in enabling “off-the-shelf” allogeneic cellular immunotherapies.

Examples of the above-described embodiments may also include the following:
1. An RNA interference (RNAi) DNA oligonucleotide for inhibiting expression of HLA A, B and C.
2. The RNAi DNA oligonucleotide of example 1, wherein the oligonucleotide comprises siRNA or shRNA.
3. The RNAi DNA oligonucleotide of examples 1 or 2, wherein the oligonucleotide does not inhibit expression of HLA-E.
4. The RNAi DNA oligonucleotide of examples 1-3, wherein the oligonucleotide is shRNA.
5. The RNAi DNA oligonucleotide of examples 1-4, wherein the oligonucleotide is 20-25 base pairs long.
6. The RNAi DNA oligonucleotide of examples 1-5, wherein the oligonucleotide comprises at least two mismatches.
7. The RNAi DNA oligonucleotide of examples 1-6, wherein the oligonucleotide comprises three mismatches.
8. The RNAi DNA oligonucleotide of examples 1-7, wherein the mismatches are with an allele comprising HLA-A, HLA-B, HLA-C, or HLA-E.
9. An RNAi DNA oligonucleotide means for inhibiting expression of HLA-A, HLA-B or HLA-C.
10. The RNAi DNA oligonucleotide of examples 1-9, wherein the mismatch is with an HLA-E allele.
11. The RNAi DNA oligonucleotide of examples 1-10, wherein the RNAi DNA oligonucleotide comprises a nucleotide sequence of SEQ ID NO. 1-30 and its complement.
12. The RNAi DNA oligonucleotide of examples 1-11, wherein the RNAi DNA oligonucleotide comprises a nucleotide sequence of SEQ ID NO: 1 or SEQ ID NO: 13, and its complement.
13. A vector comprising the RNAi DNA oligonucleotide of examples 1-12.
14. The vector of example 13 wherein the vector is a lentiviral vector.
15. The vector of examples 13-14, wherein the RNAi DNA oligonucleotide is operably linked to a promoter.
16. The vector of example 15, wherein the promoter comprises an RNA polymerase III or an EF-1α promoter.
17. The vector of examples 13-16, wherein the vector further comprises a nucleotide sequence encoding HLA-E or variants thereof.
18. The vector of example 17, wherein the HLA-E sequence comprises a mutation in the heavy chain.
19. The vector of example 18, wherein the mutation comprises Y84A or Y84C.
20. The vector of example 13-19 wherein the vector further comprises a nucleotide sequence encoding PD-L1.
21. A vector means for inhibiting expression of HLA-A, HLA-B and HLA-C.
22. A host cell transduced by the vector of examples 13-21.
23. The host cell of example 20, wherein the cell is a T cell, a Natural Killer (NK) cell, a macrophage, or an induced pluripotent stem cell.
24. A host cell means for increased CAR cytotoxicity.
25. A method of making an allogenic chimeric antigen receptor (CAR) immune cell resistant to host rejection comprising transducing an immune cell with the vector of examples 13-21.
26. The method of example 25, wherein the vector further comprises a nucleotide sequence encoding HLA-E or variants thereof.
27. The method of examples 25-26, wherein the vector further comprises a nucleotide sequence encoding PD-L1.
28. The method of examples 25-27, wherein the vector comprises the nucleotide sequence of SEQ ID NO. 1 or SEQ ID NO: 13, and its complement, a nucleotide sequence encoding HLA-E or variants thereof, or a nucleotide sequence encoding PD-L1, operably linked to one or more heterologous promoter.
29. The method of example 25, wherein the immune cell is a T cell, an NK cell, a macrophage, or an induced pluripotent stem cell.
30. A method of making an allogenic cell resistant to host rejection comprising transducing the cell with a vector comprising a nucleotide sequence encoding at least one of HLA-E or variant thereof, or PD-L1.
31. A method for increasing cytotoxicity of a chimeric antigen receptor (CAR) cell.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield essentially similar results.

What is claimed:

1. An RNA interference (RNAi) DNA oligonucleotide for inhibiting expression of HLA A, B and C.

2. The RNAi DNA oligonucleotide of claim 1, wherein the oligonucleotide comprises siRNA or shRNA.

3. The RNAi DNA oligonucleotide of claim 1, wherein the oligonucleotide does not inhibit expression of HLA-E.

4. The RNAi DNA oligonucleotide of claim 1, wherein the oligonucleotide is 20-25 base pairs long.

5. The RNAi DNA oligonucleotide of claim 1, wherein the oligonucleotide comprises at least two mismatches.

6. The RNAi DNA oligonucleotide of claim 1, wherein the oligonucleotide comprises three mismatches.

7. The RNAi DNA oligonucleotide of claim 1, wherein the mismatches are with an allele comprising HLA-A, HLA-B, HLA-C, or HLA-E.

8. The RNAi DNA oligonucleotide of claim 1, wherein the mismatch is with an HLA-E allele.

9. The RNAi DNA oligonucleotide of claim 1, wherein the RNAi DNA oligonucleotide comprises a nucleotide sequence of SEQ ID NO. 1-30 and its complement.

10. The RNAi DNA oligonucleotide of claim 1, wherein the RNAi DNA oligonucleotide comprises a nucleotide sequence of SEQ ID NO: 1 or SEQ ID NO: 13, and its complement.

11. A vector comprising the RNAi DNA oligonucleotide of claim 1.

12. The vector of claim 11 wherein the vector is a lentiviral vector.

13. The vector of claim 12, wherein the vector further comprises a nucleotide sequence encoding HLA-E or variants thereof.

14. The vector of claim 13, wherein the HLA-E sequence comprises a mutation in the heavy chain.

15. The vector of claim 13, wherein the mutation comprises Y84A or Y84C.

16. The vector of claim 11 wherein the vector further comprises a nucleotide sequence encoding PD-1.

17. A host cell transduced by the vector of claim 11.

18. The host cell of claim 17, wherein the cell is a T cell, a Natural Killer (NK) cell, a macrophage, or an induced pluripotent stem cell.

19. A method of making an allogenic chimeric antigen receptor (CAR) immune cell resistant to host rejection comprising transducing an immune cell with the vector of claim 11.

20. A method of making an allogenic cell resistant to host rejection comprising transducing the cell with a vector comprising a nucleotide sequence encoding at least one of HLA-E or variant thereof, or PD-1.
